SEQUENCE LISTING (9 sequences)

SEQ ID NO: 1 - protein, 609 aa, synthetic construct
MGSVTPIPLPKIDEPEEYNTNYILFWNHVGLELNRVTHTVGGPLTGPPLSARALGMLHLAIHDAYFSICPPTDFTTFLSPDTENAAYRLPSPNGANDARQAVAGAALKMLSSLYMKPVEQPNPNPGANISDNAYAQLGLVLDRSVLEAPGGVDRESASFMFGEDVADVFFALLNDPRGASQEGYHPTPGRYKFDDEPTHPVVLIPVDPNNPNGPKMPFRQYHAPFYGKTTKRFATQSEHFLADPPGLRSNADETAEYDDAVRVAIAMGGAQALNSTKRSPWQTAQGLYWAYDGSNLIGTPPRFYNQIVRRIAVTYKKEEDLANSEVNNADFARLFALVDVACTDAGIFSWKEKWEFEFWRPLSGVRDDGRPDHGDPFWLTLGAPATNTNDIPFKPPFPAYPSGHATFGGAVFQMVRRYYNGRVGTWKDDEPDNIAIDMMISEELNGVNRDLRQPYDPTAPIEDQPGIVRTRIVRHFDSAWELMFENAISRIFLGVHWRFDAAAARDILIPTTTKDVYAVDNNGATVFQNVEDIRYTTRGTREDEEGLFPIGGVPLGIEIADEIFNNGLKPTPPEIQPMPQETPVQKPVGQQPVKGMWEEEQAPVVKEAP

SEQ ID NO: 2 - protein, 609 aa, synthetic construct
MGSITPIPLPKIDEPEEYNTNYILFWNHVGLELNRVTHTVGGPLTGPPLSARALGMLHLAIHDAYFSIYPPTDFSTFLSPNAENAAYRLPSPNGANDARQAVAGAALKMLTSLYMKPVETPNPNPGANISDNAYAQLALVIDRSVLKAPGGVDRESASFMFGETVADVFFALLNDPRGASQEGYHPTPGRYKFDDEPTHPVVLIPVDPNNPNGPKKPFRQYHAPFYGKTTKRFATQSEHFLADPPGLRSNADETAEYDDAIRVAIAMGGAQALNSTKRSPWQTAQGLFWAYDGSNLIGTPPRFYNQIVRRIAVTYKKEEDLANSEVNNADFARLFALVDVACTDAGIFSWKEKWEYEFWRPLSGVRDDGRPDHGDPFWLTLGAPATNTNDIPFKPPFPAYPSGHATFGGAVFQMVRRYYNGRVGTWKDDEPDNIAIDMMISEELNGLNRDLRQPYDPTAPIEDQPGIVRTRIVRHFDSAWELMFENAISRIFLGVHWRFDAAAARDILIPTTTKDVYAVDNNGATVFQNVEDIRYTTKGTREDREGLFPIGGVPLGIEIADEIFNNGLKPTPPELQPMPQQTPVQKPVGQQPVQGMWAEEQAPVIKEAP

SEQ ID NO: 3 - protein, 604 aa, synthetic construct
MGSVTPIPIPRIDEPEEYNTNYILFWNHVGLELNRITHTVGGPLTGPPLSARALGMLHLAIHDAYFSICPPSDFTTFLSPDAENVAYRLPSLNGADDARQAVAGAALKMLSLLYMKPVEQPNPNPGANISDNAYAQLGLVLDRSVLKAPGGVDQESASFMFGEAVADVFFALLNDPRGASQEGYHPTLGRYKFDDEPTHPVVLIPVDPNNPNGPKKPFRQYHAPFYGKTTKRFATQSEHFLADPPGLRSNADETAEYDDSIRVAIAMGGAQGLNSTKRSPWQTAQGLYWAYDGSNLIGTPPRFYNQIVRRIAVTYKKEEDLANSEVNNADFARLFALVNVACTDAGIFSWKEKWEFEFWRPLSGVRDDGRPDHGDPFWLTLGAPATNTNDIPFKPPFPAYPSGHATFGGAVFQMVRRYYNGRVGTWKDDEPDNIAIDMMISEELNGLNRDLRQPYDPTAPIEDQPGIVRTRIVRHFDSAWEMMFENAISRIFLGVHWRFDAAAARDILIPTTTKDVYAVDNNGATVFQNVEDVRYSTKGTREGREGLFPIGGVPLGIEIADEIFNNGLRPTPPELQPMPQETPVQKPVEGMWEEEQAPIVNEAP

SEQ ID NO: 4 - protein, 604 aa, synthetic construct
MAAITPIPLPKIDEPEEYNTNYILYWNHVGLELNRVTHTVGGPLTGPPLSARALGMLHLAIHDAYFSIHPSADFHTFLSPDATDPAYRLPTPQGANDARQAVAGASLKMLTSLYSKPTVQPNPNPGANISDNAYAQLGLVLDRSALKAPGGVDRASASFSFGEAVADVFFALLNDPRGASQEGYHPTPGRYKFDDEPTHPVTLIPVDPNNPNGPKKPFRQYHAPFYGKTTKRLATQTEHMLADPPGLRSNANETAEYDDAVRVAIAMGGAQGLNSTKRSPWQTVQGMYWAYDGSNLIGTPPRFYNQIVRRIAVTYKKEADLANSEVNNADFARLFALVDVACADAGIFSWKEKWEFEFWRPLSGVRDDGRPDHADPFWLTLGAPSTNTNDIPFKPPFPAYPSGHATFGGAVFQMVRRYYNGRVGAWKNDEIDNIAIDMMVSEELNGLNRDLRQPYDPTAPIEDQPGIVRTRITRHFGSAWELMFENAISRIFLGVHWRFDAAAARDILIPTTTKDVYAVDGTGATVFQNIEDVKYTTRGTRQGCEGLYPIGGVPLGMEIANEIFESGLRPTPAERQPKVKEADMQETVGGMWEGEEAPLVNEAP

SEQ ID NO: 5 - protein, 607 aa, synthetic construct
MGSVIPIPLPKIDEPEEYNTNYVLFWNNVGLELNRVTHTVGGPLTGPPLSARALGMLHLAIHDAYFSIYPPTDFTTFLSPDAENAAYRLPSLNGANDARQAVAGAALKMLSSLYMKPDSSSGGSISDNAYAQLALVIDRSVLKAPGGVDRESASFVFGEAVADVFFALLNDPRGASQEGYHPTPGRYKFDDEPTHPVVLVPVDPNNPNGPKKPFRQYHAPFYGKTTKRFATQTEHFLADPPGLRSNADETAEYDDSIRVAVAMGGAQGLNSTKRSPWQTAQGLFWAYDGSNLVGTPPRFYNQIVRRIAVTYKKEEDLANSEVNNADFARLFALVDVACTDAGIFSWKEKWEYEFWRPLSGVRDDGRPDHGDPFWLTLGAPATNTNDIPFKPPFPAYPSGHATFGGAVFQMVRRYYNGRVGTWKDDEPDNIAIDMVISEELNGLNRDLRQPYDPTAPIEDQPGIVRTRIVRHFDSAWEMMFENAISRIFLGVHWRFDAAAARDILIPTTTKDVYAVDNNGATVFQNVEDIRYSTKGTREDREGLFPIGGVPLGIEIADEIFNNGLRPTPPELQPMPQETPVQKPIGQQLVQGLWHDEEGQVPVVKEAP

SEQ ID NO: 6 - protein, 609 aa, synthetic construct
MGSVTPIPLPRIDEPEEYNTNYILFWNNAGLELNRVTHTVGGPLTGPPLSARALGMLHLAIHDAYFSIFPPSDFTTFLSPDAENAAYRLPSLNGADDARQAVAGAALKMLSLLYMKPIEQPNPNPGANISDNAYAQLGLVLDRSALKAPGGVDRESVSFMFGEAVADVFFALLNDPRGASQEGYHPTPGRYKFDDEPTHPVVLIPVDPNNPSGPKMPFRQYHAPFYGKTTKRFATQSEHFLADPPGLRSNADETAEYDDSIRTAIAMGGAPGLNSTKRSPWQTAQGLYWAYDGSNLIGTPPRFYNQIVRRIAVTYKKEEDLANSEVNNADFARLFALVDVACTDAGIFSWKEKWEFEFWRPLSGVRDDGRPDHGDPTWLTLGAPATNTNDIPFKPPFPAYPSGHATFGGAVFQMVRRYYNGRVGTWKDDEPDNIAIDMMISEELNGLNRDLRQPYDPTAPIEDQPGIVRTRIVRHFDSAWELMFENAISRIFLGVHWRFDAAAARDILIPTTTKDVYAVDNNGATIFQNVEDIRYSTKGTREDREGLFPIGGVPLGIEIADEIFNNGLKPTPPELQPMPQETPVQKPVGQQPVKGMWEEEQAPIVKEAP

SEQ ID NO: 7 - protein, 604 aa, synthetic construct
MGSVTPIPLPKIDEPEEYNTNYILFWNYVGLELNRVTHTVGGPLTGPPLSARALGMLHLAVHDAYFSICPPTDFTTFLSPDAENAAYRLPSTNGANDARQAVAGAALKMLTSLYMKPIEQPNPNPSANISDNAYAQLALVLDRSAMEAPGGIDRESASFMFGEAVADVFFALLNDPRGASQEGYHPTPGRYKFDDEPTHPVVLIPVDPNNPNGPKKPFRQYHAPFYGKTTKRFATQSEHFLADPPGLRSNADETAEYDDAIRVAIAMGGAQALNSTKRSPWQTAQGLYWAYDGSNLIGTPPRFYNQIVRRIAVTYKKEEDIANSEVNNADFARLFALVDVACTDAGIFSWKEKWEFEFWRPLSGVRDDGRPDHGDPFWLTLGAPATNTNDIPFKPPFPAYPSGHATFGGAVFQMVRRYYNGRVGTWKDDEPDNIAIDMMISEELNGLNRDLRQPYDPTAPIEDQPGIVRTRIVRHFDSAWELMFENAISRIFLGVHWRFDAAAARDILIPTTTKDVYAVDNNGATVFQNVEDIRYTTKGTREDREGLFPIGGVPLGIEIANEIFENGLKPTPPEIQPMPQETPAQKPVKGMWQEEQVPVVEEAP

SEQ ID NO: 8 - protein, 607 aa, synthetic construct; identical to SEQ ID NO: 5

SEQ ID NO: 9 - protein, 609 aa, synthetic construct
MGSVTPIPLPKIDEPEEYNTNYILFWNHVGLELNRVTHTVGGPLTGPPLSARALGMLHLAIHDAYFSIXPPTDFTTFLSPDAENAAYRLPSLNGANDARQAVAGAALKMLSSLYMKPVEQPNPNPGANISDNAYAQLXLVLDRSVLKAPGGVDRESASFMFGEAVADVFFALLNDPRGASQEGYHPTPGRYKFDDEPTHPVVLIPVDPNNPNGPKKPFRQYHAPFYGKTTKRFATQSEHFLADPPGLRSNADETAEYDDXIRVAIAMGGAQGLNSTKRSPWQTAQGLYWAYDGSNLIGTPPRFYNQIVRRIAVTYKKEEDLANSEVNNADFARLFALVDVACTDAGIFSWKEKWEFEFWRPLSGVRDDGRPDHGDPFWLTLGAPATNTNDIPFKPPFPAYPSGHATFGGAVFQMVRRYYNGRVGTWKDDEPDNIAIDMMISEELNGLNRDLRQPYDPTAPIEDQPGIVRTRIVRHFDSAWELMFENAISRIFLGVHWRFDAAAARDILIPTTTKDVYAVDNNGATVFQNVEDIRYXTKGTREDREGLFPIGGVPLGIEIADEIFNNGLXPTPPELQPMPQETPVQKPXGQQXVXGMWEEEQAPVVKEAP

(19) United States
(12) Patent Application Publication
(10) Pub. No.: US 2026/0232745 A1
(43) Pub. Date: Aug. 13, 2026

(54) INHIBITION OF METHANE PRODUCTION IN RUMINANTS

(71) Applicant: Loam Bio Pty Ltd, Orange (AU)

(72) Inventors: Abed CHAUDHURY, Orange (AU); Karen PACO, St. Paul, MN (US); Tristan YANG, St. Paul, MN (US); Timothy CHAI, St. Paul, MN (US); Hop PHAN, Orange (AU); Matthew CALLAGHA, Orange (AU); Nigel TOMKINS, Orange (AU); Matthew MYERS, Orange (AU)

(21) Appl. No.: 19/152,898

(22) PCT Filed: Feb. 1, 2024
(86) PCT No.: PCT/US2024/014050

§ 371 Date: Jul. 31, 2025

Related U.S. Application Data
(60) Provisional application No. 63/482,781, filed on Feb. 1, 2023.
(60) Provisional application No. 63/585,310, filed on Sep. 26, 2023.

(51) Int. Cl. A61K 36/062; A23K 10/30; A23K 20/189; A23K 50/10; A61K 31/02; A61K 38/44; C12N 1/145; C12N 9/08; C12R 1/645

(52) U.S. Cl. CPC A61K 36/062; A23K 10/30; A23K 20/189; A23K 50/10; A61K 31/02; A61K 38/44; C12N 1/145; C12N 9/0065; C12R 2001/645

(57) ABSTRACT

The present disclosure provides a method for reducing methane emissions from a ruminant comprising administering to the ruminant a composition comprising a fungal strain, biomass from the fungal strain, a culture supernatant from the fungal strain, or a combination thereof, wherein the fungal strain comprises a vanadium-dependent haloperoxidase (VHPO) with an amino sequence having at least 80% identity with SEQ ID NO: 3, SEQ ID NO: 4, or SEQ ID NO: 5, SEQ ID NO: 6, SEQ ID NO: 7, or SEQ ID NO: 8. The disclosure also provides a method for reducing methane emissions from a ruminant comprising administering to the ruminant a composition comprising a fungal strain, biomass from the fungal strain, a culture supernatant from the fungal strain, or a combination thereof.

### CROSS REFERENCE TO RELATED APPLICATIONS

This application claims the benefit of U.S. Provisional Patent Application No. 63/482,781, filed on Feb. 1, 2023, and U.S. Provisional Patent Application No. 63/585,310, filed on Sep. 26, 2023, the contents of which are incorporated herein by reference in their entirety.

### STATEMENT REGARDING ELECTRONIC FILING OF A SEQUENCE LISTING

A Sequence Listing in XML format, submitted under 37 C.F.R. § 1.821, entitled LOAM-B002-02WO.xml, 13,963 bytes in size, generated on Feb. 1, 2024, and filed via EFS-Web, is provided in lieu of a paper copy. This Sequence Listing is incorporated by reference into the specification for its disclosures.

### TECHNICAL FIELD

The present disclosure relates to the reduction of methane emissions from ruminants. Specifically, the present invention relates to methods and processes associated with the use of fungal strains and their enzymatic production of haloforms that, when administered to ruminants, can reduce the methane production in the rumen, thereby reducing methane emissions.

### BACKGROUND

Marine macroalgae, seaweeds, are producers of halogenated natural products. Biosynthesis of halogenated molecules is linked to reactive oxygen species (ROS) such as hydrogen peroxide that are substrates for haloperoxidase enzymes that promote the formation of halogenated molecules. It has been shown that vanadium-dependent haloperoxidases (VHPOs), halogenating enzymes that are present in seaweeds, are involved in bromoform biosynthesis. The importance of bromoform synthesis is that it can inhibit methane production. The antimethanogenic activity of bromoform was studied in vitro by isolating bioactive compounds from the red seaweed Asparagopsis.

The red macroalga Asparagopsis taxiformis has been linked to the reduction of methane (CH₄) from beef cattle by up to 99%. A recent study shows that A. taxiformis is a highly efficient feed supplement for CH₄ mitigation during enteric fermentation. The antimethanogenic compounds found in A. taxiformis are: bromoform, dibromochloromethane, bromochloroacetic acid, dibromoacetic acid and dichloromethane. However, bromoform, a halomethane, is the most abundant antimethanogenic compound found in A. taxiformis. Bromoform and dibromochloromethane had the highest activity at inhibiting methane production.

Bromoform is a halogen that has been found to interfere with the methanogenesis pathway by serving as a competitive inhibitor or analog of methyl-coenzyme M reductase (MCR), preventing the final catalysis step.

It would be attractive, practically and commercially, to be able to reduce the methane emissions produced by ruminants, and in particular, domestic livestock, by using a source of bromoform that is easily produced with limited impact on the environment. Fungal strains expressing VHPOs present an alternative to marine macroalgae for accomplishing this objective.

### SUMMARY

In some aspects, the present disclosure relates to a method for reducing methane emissions from a ruminant comprising administering to the ruminant a composition comprising a fungal strain, biomass from the fungal strain, a culture supernatant from the fungal strain, or a combination thereof, wherein the fungal strain comprises a vanadium-dependent haloperoxidase (VHPO) with an amino sequence having at least 80% identity with SEQ ID NO: 3, SEQ ID NO: 4, SEQ ID NO: 5, SEQ ID NO: 6, SEQ ID NO: 7, or SEQ ID NO: 8.

In one aspect, the VHPO comprises SEQ ID NO: 3. In another aspect, the VHPO comprises SEQ ID NO: 4. In another aspect, the VHPO comprises SEQ ID NO: 5. In another aspect, the VHPO comprises SEQ ID NO: 6. In another aspect, the VHPO comprises SEQ ID NO: 7. In another aspect, the VHPO comprises SEQ ID NO: 8. In another aspect, the VHPO comprises SEQ ID NO: 9.

In certain aspects, the VHPO demonstrates an improved property compared to a reference VHPO comprising SEQ ID NO: 1 or SEQ ID NO: 2, and the improved property is increased substrate specificity, substrate selectivity, substrate binding, cofactor specificity, cofactor selectivity, cofactor binding, enzyme activity, enzyme kinetics, enzyme expression, enzyme stability, enzyme biocatalysis (i.e., titer, rate, yield, specific yield, and total turnover number), enzyme glycosylation, enzyme methylation, enzyme acetylation, retention of the enzyme in the cell or sub-cellular component, secretion of the enzyme from the cell, and/or enzyme activity across a broader temperature range or pH range compared to the reference VHPO. Titer refers to the mass product/reaction volume and is also termed product concentration. Rate refers to the mass product/reaction volume/reaction time and is also termed productivity or space-time yield.

In other aspects, the present disclosure relates to a method for reducing methane emissions from a ruminant comprising administering to the ruminant a halomethane produced with a vanadium-dependent haloperoxidase (VHPO) having an amino sequence with at least 80% identity with SEQ ID NO: 3, SEQ ID NO: 4, SEQ ID NO: 5, SEQ ID NO: 6, SEQ ID NO: 7, or SEQ ID NO: 8, wherein the halomethane is selected from the group consisting of methyl bromide, methylene bromide, bromoform, methyl iodide, methylene iodide, iodoform, methyl chloride, methylene chloride, chloroform, and combinations thereof. In one aspect, the present disclosure relates to a method for reducing methane emissions from a ruminant comprising administering to the ruminant a halomethane produced with a vanadium-dependent haloperoxidase (VHPO) having an amino sequence with at least 80% identity with SEQ ID NO: 9, wherein the halomethane is selected from the group consisting of methyl bromide, methylene bromide, bromoform, methyl iodide, methylene iodide, iodoform, methyl chloride, methylene chloride, chloroform, and combinations thereof.

In one aspect, the halomethane is bromoform. In another aspect, halomethane is administered in an amount of between 1 mg and 1000 mg per day to the ruminant. In other aspects, the halomethane is administered to the ruminant in an amount of between 1 mg and 900 mg per day, between 1 mg and 800 mg per day, between 1 mg and 700 mg per day, between 1 mg and 600 mg per day, between 1 mg and 500 mg per day, between 1 mg and 400 mg per day, between 1 mg and 300 mg per day, between 1 mg and 200 mg per day, or between 1 mg and 100 mg per day.

In another aspect, the halomethane is administered in an amount of between 0.01% and 10.0% dry matter intake (DMI) dosage, between 0.01% and 5.0% DMI dosage, between 0.01% and 1.0% DMI dosage, between 0.01% and 0.5% DMI dosage, between 0.1% and 10.0% DMI dosage, between 0.10% and 5.0% DMI dosage, between 0.10% and 1.0% DMI dosage, or between 0.1% and 0.5% DMI dosage.

In another aspect, the halomethane is administered in an amount of between 1 mg/kg DMI and 1000 mg/kg DMI, between 1 mg/kg DMI and 900 mg/kg DMI, between 1 mg/kg DMI and 800 mg/kg DMI, between 1 mg/kg DMI and 700 mg/kg DMI, between 1 mg/kg DMI and 600 mg/kg DMI, between 1 mg/kg DMI and 500 mg/kg DMI, between 1 mg/kg DMI and 400 mg/kg DMI, between 1 mg/kg DMI and 300 mg/kg DMI, between 1 mg/kg DMI and 200 mg/kg DMI, between 1 mg/kg DMI and 100 mg/kg DMI, or between 1 mg/kg DMI and 50 mg/kg DMI.

In another aspect, the halomethane is administered to a ruminant in an amount of between 0.01 mg/kg/day and 250 mg/kg/day, 0.01 mg/kg/day and 200 mg/kg/day, 0.01 mg/kg/day and 150 mg/kg/day, 0.01 mg/kg/day and 100 mg/kg/day, 0.01 mg/kg/day and 50 mg/kg/day, or 0.01 mg/kg/day and 10 mg/kg/day. In one aspect, the halomethane is administered to a ruminant in an amount of between 0.1 mg/kg/day and 250 mg/kg/day, 0.1 mg/kg/day and 200 mg/kg/day, 0.1 mg/kg/day and 150 mg/kg/day, 0.1 mg/kg/day and 100 mg/kg/day, 0.1 mg/kg/day and 50 mg/kg/day, or 0.1 mg/kg/day and 10 mg/kg/day.

In some aspects, the present disclosure relates to a method for reducing methane emissions from a ruminant comprising administering to the ruminant a composition comprising a fungal strain, biomass from the fungal strain, a culture supernatant from the fungal strain, or a combination thereof, wherein the fungal strain is Curvularia lunata US-991 (ATCC Accession No. PTA-127504), Curvularia pseudobrachyspora US-215 (ATCC Accession No. PTA-127509), Curvularia spicifera US-188 (ATCC Accession No. PTA-127510), Curvularia inaequalis US-998 (ATCC Accession No. PTA-127511), Curvularia intermedia US-429 (ATCC Accession No. PTA-127512), Curvularia hawaiiensis US-446 (ATCC Accession No. PTA-127601), Curvularia protuberata US-1111 (ATCC Accession No. PTA-127607), or a mutant thereof having all identifying characteristics of the strain.

In one aspect, the fungal strain is Curvularia lunata US-991 (ATCC Accession No. PTA-127504) or a mutant thereof having all identifying characteristics of the strain. In another aspect, the fungal strain is Curvularia pseudobrachyspora US-215 (ATCC Accession No. PTA-127509) or a mutant thereof having all identifying characteristics of the strain. In another aspect, the fungal strain is Curvularia spicifera US-188 (ATCC Accession No. PTA-127510) or a mutant thereof having all identifying characteristics of the strain. In another aspect, the fungal strain is Curvularia inaequalis US-998 (ATCC Accession No. PTA-127511) or a mutant thereof having all identifying characteristics of the strain. In another aspect, the fungal strain is Curvularia intermedia US-429 (ATCC Accession No. PTA-127512) or a mutant thereof having all identifying characteristics of the strain. In another aspect, the fungal strain is Curvularia hawaiiensis US-446 (ATCC Accession No. PTA-127601) or a mutant thereof having all identifying characteristics of the strain. In another aspect, the fungal strain is Curvularia protuberata US-1111 (ATCC Accession No. PTA-127607) or a mutant thereof having all identifying characteristics of the strain.

In some aspects, the present disclosure relates to a method for reducing methane emissions from a ruminant comprising administering to the ruminant a halomethane produced by a fungal strain selected from the group consisting of Curvularia lunata US-991 (ATCC Accession No. PTA-127504), Curvularia pseudobrachyspora US-215 (ATCC Accession No. PTA-127509), Curvularia spicifera US-188 (ATCC Accession No. PTA-127510), Curvularia inaequalis US-998 (ATCC Accession No. PTA-127511), Curvularia intermedia US-429 (ATCC Accession No. PTA-127512), Curvularia hawaiiensis US-446 (ATCC Accession No. PTA-127601), Curvularia protuberata US-1111 (ATCC Accession No. PTA-127607), and a mutant thereof having all identifying characteristics of the strain, wherein the halomethane is selected from the group consisting of methyl bromide, methylene bromide, bromoform, methyl iodide, methylene iodide, iodoform, methyl chloride, methylene chloride, chloroform, and combinations thereof.

In certain aspects, the ruminant is a member of the family Bovidae. In one aspect, the ruminant is Bos taurus or Bos indicus. In another aspect, the ruminant is Ovis aries or Capra hircus.

In yet other aspects, the present disclosure relates to a cell or a biologically pure culture of a fungal strain comprising a VHPO with an amino sequence having at least 80% identity with SEQ ID NO: 3, SEQ ID NO: 4, SEQ ID NO: 5, SEQ ID NO: 6, SEQ ID NO: 7, or SEQ ID NO: 8.

In one aspect, the fungal strain is a yeast belonging to the genus of Arxula, Candida, Ogataea, Kluyveromyces, Pichia, Saccharomyces, or Yarrowia. In another aspect, the yeast is Saccharomyces cerevisiae, Yarrowia lipolytica, or Pichia pastoris.

In yet other aspects, the present disclosure relates to a cell or a biologically pure culture of Curvularia lunata US-991 (ATCC Accession No. PTA-127504), Curvularia pseudobrachyspora US-215 (ATCC Accession No. PTA-127509), Curvularia spicifera US-188 (ATCC Accession No. PTA-127510), Curvularia inaequalis US-998 (ATCC Accession No. PTA-127511), Curvularia intermedia US-429 (ATCC Accession No. PTA-127512), Curvularia hawaiiensis US-446 (ATCC Accession No. PTA-127601), Curvularia protuberata US-1111 (ATCC Accession No. PTA-127607), or a mutant thereof having all identifying characteristics of the strain.

In one aspect, the present disclosure provides an agricultural composition comprising a cell or a biologically pure culture disclosed herein or biomass from the cell or biologically pure culture, a culture supernatant from the cell or biologically pure culture, or a combination thereof, and, optionally, an agriculturally acceptable carrier.

In certain aspects, the agriculturally acceptable carrier is a protective hydrocolloid, such as a gums, protein, modified starch, binder, film-forming agent, encapsulating agent/material, wall/shell material, matrix compound, coating, emulsifier, foaming agent, surface active agent, solubilizing agent, e g., oil, fat, wax, lecithin, etc., adsorbent, filler, co-compounds, dispersing agent, wetting agent, processing aid (solvent), flowing agent, flavoring agent, sweetening agent, coloring agent, weighting agent, jellifying agent, gel-forming agent, anti-oxidant, anti-microbial or other preservative agent. The agricultural composition may be in any galenic formulation that is suitable for administrating to the ruminant, especially in any form that is conventional for oral administration, e.g., in solid forms, such as (additives/supplements for) food or feed, food or feed premix, fortified food or feed, tablets, seed, pills, granules, capsules, or in liquid forms, such as solutions, emulsions or suspensions, e.g., liquids, pastes, and oily suspensions. The pastes may be filled into hard- or soft-shell capsules. The agricultural compositions may be in the form of controlled immediate or sustained-release formulations.

In some aspects, the agricultural composition further comprises a cereal, starch, vegetable waste, vitamin, mineral, trace element, emulsifier, aromatizing product, binder, colorant, odorant, thickening agent, or a combination thereof.

In yet other aspects, the present disclosure provides animal feed comprising a biologically pure culture or an agricultural composition disclosed herein.

In some aspects, the present disclosure relates to the use of a cell or a biologically pure culture, an agricultural composition, or animal feed disclosed herein for reducing methane emissions from a ruminant.

The following description is intended only by way of example, and simply illustrates certain selected embodiments of devices, systems, and processes that are consistent with the disclosed subject matter as claimed herein.

### BRIEF DESCRIPTION OF THE DRAWINGS

FIG. 1 depicts a multiple sequence alignment with the VHPO amino acid sequences from Curvularia inaequalis PDB ID 1VNC (SEQ ID NO: 1), Curvularia sp. 4388 (NMI Accession No. V22/011149) (SEQ ID NO: 2), Curvularia spicifera US-188 (ATCC Accession No. PTA-127510) (SEQ ID NO: 3), Curvularia intermedia US-429 (ATCC Accession No. PTA-127512) (SEQ ID NO: 4), Curvularia pseudobrachyspora US-215 (ATCC Accession No. PTA-127509) (SEQ ID NO: 5), Curvularia hawaiiensis US-446 (ATCC Accession No. PTA-127601) (SEQ ID NO: 6), Curvularia protuberata US-1111 (ATCC Accession No. PTA-127607) (SEQ ID NO: 7), and Curvularia lunata US-991 (ATCC Accession No. PTA-127504) (SEQ ID NO: 8).

FIG. 2 depicts the predicted protein structure for the VHPO enzyme expressed by Curvularia intermedia US-429 (ATCC Accession No. PTA-127512) along with alignments of this predicted structure with those of Curvularia inaequalis PDB ID 1VNC and Curvularia sp. 4388 (NMI Accession No. V22/011149).

FIG. 3 depicts the predicted protein structure for the VHPO enzyme expressed by Curvularia pseudobrachyspora US-215 (ATCC Accession No. PTA-127509) along with alignments of this predicted structure with those of Curvularia inaequalis PDB ID 1VNC and Curvularia sp. 4388 (NMI Accession No. V22/011149).

FIG. 4 depicts the predicted protein structure for the VHPO enzyme expressed by Curvularia spicifera US-188 (ATCC Accession No. PTA-127510) along with alignments of this predicted structure with those of Curvularia inaequalis PDB ID 1VNC and Curvularia sp. 4388 (NMI Accession No. V22/011149).

FIG. 5 depicts the VHPO reaction mechanism.

FIGS. 6A-6F depicts a superposition of the reference structure of each VHPO enzyme to that of Curvularia sp. 4388 (NMI Accession No. V22/011149) (i.e., SEQ ID NO: 2). FIGS. 6A, 6B, 6C, 6D, 6E, and 6F present SEQ ID NO: 1, SEQ ID NO: 3, SEQ ID NO: 4, SEQ ID NO: 5, SEQ ID NO: 6, and SEQ ID NO: 7, respectively. The reference structures are the lowest mean-square deviation structure to the average structure of the simulations.

FIG. 7 depicts VHPO active center. Distances are shown in A.

FIG. 8 depicts a comparison of active centers from SEQ ID NOs: 1-7.

FIG. 9 depicts the active center pocket sizes for SEQ ID NO: 1 and SEQ ID NO: 2.

FIGS. 10A, 10B, 10C, and 10D depict the pH profile of VHPO activity measured using the MCD assay for the VHPO from Curvularia sp. 4388 (NMI Accession No. V22/011149) (i.e., SEQ ID NO: 2), the VHPO from Curvularia spicifera US-188 (ATCC Accession No. PTA-127510) (i.e., SEQ ID NO: 3), the VHPO from Curvularia pseudobrachyspora US-215 (ATCC Accession No. PTA-127509) (i.e., SEQ ID NO: 5), and the VHPO from Curvularia protuberata US-1111 (ATCC Accession No. PTA-127607) (i.e., SEQ ID NO: 7), respectively.

FIGS. 11A, 11B, 11C, 11D, and 11E depict VHPO kinetic activity against KBr concentrations with Curvularia sp. 4388 (NMI Accession No. V22/011149) culture supernatant at pH 5.5, purified VHPO from Curvularia sp. 4388 (NMI Accession No. V22/011149) (i.e., SEQ ID NO: 2) at pH 5.5, purified VHPO from Curvularia spicifera US-188 (ATCC Accession No. PTA-127510) (i.e., SEQ ID NO: 3) at pH 6.5, purified VHPO from Curvularia pseudobrachyspora US-215 (ATCC Accession No. PTA-127509) (i.e., SEQ ID NO: 5) at pH 5.5, and purified VHPO from Curvularia protuberata US-1111 (ATCC Accession No. PTA-127607) (i.e., SEQ ID NO: 7) at pH 5.5, respectively.

FIGS. 12A, 12B, 12C, 12D, and 12E depict VHPO kinetic activity against H₂O₂ concentrations with Curvularia sp. 4388 (NMI Accession No. V22/011149) culture supernatant at pH 5.5, purified VHPO from Curvularia sp. 4388 (NMI Accession No. V22/011149) (i.e., SEQ ID NO: 2) at pH 5.5, purified VHPO from Curvularia spicifera US-188 (ATCC Accession No. PTA-127510) (i.e., SEQ ID NO: 3) at pH 6.5, purified VHPO from Curvularia pseudobrachyspora US-215 (ATCC Accession No. PTA-127509) (i.e., SEQ ID NO: 5) at pH 5.5, and purified VHPO from Curvularia protuberata US-1111 (ATCC Accession No. PTA-127607) (i.e., SEQ ID NO: 7) at pH 5.5, respectively.

### DETAILED DESCRIPTION

Preferred features, embodiments and variations of the invention may be discerned from the following detailed description which provides sufficient information for those skilled in the art to perform the invention. The detailed description is not to be regarded as limiting the scope of the preceding summary of the invention in any way.

In compliance with the statute, the invention has been described in language more or less specific to structural or methodical features. The term “comprises” and its variations, such as “comprising” and “comprised of” is used throughout in an inclusive sense and not to the exclusion of any additional features. It is to be understood that the invention is not limited to specific features shown or described since the means herein described comprises preferred forms of putting the invention into effect. The invention is, therefore, claimed in any of its forms or modifications within the proper scope of the appended claims appropriately interpreted by those skilled in the art.

Throughout the specification and claims (if present), unless the context requires otherwise, the term “substantially” or “about” will be understood to not be limited to the value for the range qualified by the terms.

It is to be understood that unless specifically stated otherwise, references to “a,” “an,” and/or “the” may include one or more than one and that reference to an item in the singular may also include the item in the plural. Reference to an element by the indefinite article “a,” “an” and/or “the” does not exclude the possibility that more than one of the elements are present, unless the context clearly requires that there is one and only one of the elements. As used herein, the term “comprise,” and conjugations or any other variation thereof, are used in its non-limiting sense to mean that items following the word are included, but items not specifically mentioned are not excluded.

A “ruminant” is a mammal of the order Artiodactyla that digests plant-based food by initially softening and partially fermenting it within the animal's first stomach chambers, then regurgitating the semi-digested mass, now known as cud, and chewing it again. The process of rechewing the cud to further break down plant matter and stimulate digestion is called “ruminating”. Ruminants have a digestive tract with four chambers, namely the rumen, reticulum, omasum and abomasum. In the first two chambers, the rumen and the reticulum, the food is mixed with saliva and separates into layers of solid and liquid material. Solids clump together to form the cud, or bolus. The cud is then regurgitated, chewed slowly to completely mix it with saliva, which further breaks down fibers. Fiber, especially cellulose, is broken down into glucose in these chambers by symbiotic anaerobic bacteria, protozoa and fungi. The broken-down fiber, which is now in the liquid part of the contents, then passes through the rumen into the next stomach chamber, the omasum. The food in the abomasum is digested much like it would be in the monogastric stomach. Digested gut contents are finally sent to the small intestine, where the absorption of the nutrients occurs. Almost all the glucose produced by the breaking down of cellulose is used by the symbiotic bacteria. Ruminants get their energy from the volatile short chain fatty acids (VFAs) produced by the bacteria, namely acetate, propionate, butyrate, valerate, and isovalerate. Ruminants included cattle, goats, sheep, giraffes, yaks, deer, antelope, and others.

As used herein, the term “bovid” includes any member of the family Bovidae, which include hoofed mammals such as antelope, sheep, goats, and cattle, among others.

As used herein, the term “reducing” includes the reduction of amount of substance in comparison with a reference. For example, the reduction in the amount of total gas and/or methane produced by a ruminant animal or animals administered a composition comprising a fungal strain according to the present invention, relative to an animal or animals not administered a composition comprising a fungal strain of the present invention. The reduction can be measured in vitro with an artificial rumen system that simulates anaerobic fermentation, or in vivo with animals confined in respiration chambers. It is within the knowledge and skill of those trained in the art to assess enteric methanogenesis by a ruminant animal.

As used herein, the term “reducing methane production” refers to the reduction of methane produced in the gastro-intestinal tract. The term includes the specific volume of methane generated as a result of anaerobic fermentation, for example, in the systems described herein. Fermentation in the rumen and the gut of a ruminant gives rise to production of methane. The present invention aims to reduce this process, such as to reduce the total amount of methane produced in the gastro-intestinal tract. It is within the knowledge and skill of those trained in the art to assess methane production by a ruminant animal.

The term “specific activity” as used herein is defined as the units of activity in a given amount of protein. Thus, the specific activity is not directly measured but is calculated by dividing 1) the activity in units/ml of the enzyme sample by 2) the concentration of protein in that sample, so the specific activity is expressed as units/mg, where an enzyme unit is defined as moles of product formed/minute. The specific activity of a sample of pure, fully active enzyme is a characteristic of that enzyme. The specific activity of a sample of a mixture of proteins is a measure of the relative fraction of protein in that sample that is composed of the active enzyme of interest.

The terms “k_{cat}” and “K_{M}” are known to those skilled in the art and are described in Enzyme Structure and Mechanism, 2nd ed. (Ferst; W.H. Freeman Press, NY, 1985; pp 98-120). K_{M}, the Michaelis constant, is the concentration of substrate that leads to half-maximal velocity. The term “k_{cat}”, often called the “turnover number”, is defined as the maximum number of substrate molecules converted to products per active site per unit time, or the number of times the enzyme turns over per unit time. k_{cat}=Vₘₐₓ/[E], where [E] is the enzyme concentration (Ferst, supra). The terms “total turnover” and “total turnover number” are used herein to refer to the amount of product formed by the reaction of an enzyme with substrate.

The term “catalytic efficiency” is defined as the k_{cat}/K_{M} of an enzyme. Catalytic efficiency is used to quantify the specificity of an enzyme for a substrate.

### Fungal Strains Expressing a Vanadium-Dependent Haloperoxidase

In certain aspects, the present disclosure provides a method for reducing methane emissions from a ruminant comprising administering to the ruminant a composition comprising a fungal strain, biomass from the fungal strain, a culture supernatant from the fungal strain, or a combination thereof, wherein the fungal strain comprises a vanadium-dependent haloperoxidase (VHPO).

In some aspects, the amino acid sequence of the VHPO comprises an amino sequence having at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity with SEQ ID NO: 3, SEQ ID NO: 4, SEQ ID NO: 5, SEQ ID NO: 6, SEQ ID NO: 7, SEQ ID NO: 8, or SEQ ID NO: 9. (see Table 1). In one aspect, the VHPO comprises or consists of SEQ ID NO: 3, SEQ ID NO: 4, SEQ ID NO: 5, SEQ ID NO: 6, SEQ ID NO: 7, SEQ ID NO: 8, or SEQ ID NO: 9.

In other aspects, the amino acid sequence of the VHPO comprises an amino sequence having a tyrosine at position 69, a tyrosine at position 356, a phenylalanine at position 289, or a combination thereof. In one aspect, the VHPO comprises an amino sequence having a tyrosine at position 69, a tyrosine at position 356, and a phenylalanine at position 289.

In other aspects, the VHPO is a variant protein of the protein comprising or consisting of the amino acid sequence of SEQ ID NO: 3, SEQ ID NO: 4, SEQ ID NO: 5, SEQ ID NO: 6, SEQ ID NO: 7, SEQ ID NO: 8, or SEQ ID NO: 9. Variant proteins are within the scope of the invention as long as the resulting variant protein retains similar characteristics when compared to the parent peptide. Exemplary modifications are for example conservative substitutions that will result in VHPO variants with similar characteristics to those of the parent molecules. Conservative replacements are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. Alternatively, the amino acid repertoire can be grouped as (1) acidic (aspartate, glutamate); (2) basic (lysine, arginine, histidine), (3) aliphatic (glycine, alanine, valine, leucine, isoleucine, serine, threonine), with serine and threonine optionally grouped separately as aliphatic-hydroxyl; (4) aromatic (phenylalanine, tyrosine, tryptophan); (5) amide (asparagine, glutamine); and (6) sulfur-containing (cysteine and methionine) (Stryer (ed.), Biochemistry, 2nd ed, WH Freeman and Co., 1981). Non-conservative substitutions can be made to the VHPO variants that involve substitutions of amino acid residues between different classes of amino acids to improve properties of the VHPO variants. Whether a change in the amino acid sequence of a polypeptide or fragment thereof results in a functional homolog can be readily determined by assessing the ability of the modified polypeptide or fragment to produce a response in a fashion similar to the unmodified polypeptide or fragment using the assays described herein. Peptides, polypeptides or proteins in which more than one replacement takes place can readily be tested in the same manner.

In one aspect, the VHPO comprises an amino sequence having an aromatic amino acid at position 69. In another aspect, the VHPO comprises an amino sequence having a sulfur-containing amino acid at position 69. In another aspect, the VHPO comprises an amino sequence having a basic amino acid at position 69.

In one aspect, the VHPO comprises an amino sequence having an aliphatic-hydroxyl amino acid at position 72.

In another aspect, the VHPO comprises an amino sequence having a non-polar amino acid at position 92. In one aspect, the non-polar amino acid at position 92 is proline. In another aspect, the non-polar amino acid at position 92 is leucine.

In one aspect, the VHPO comprises an amino sequence having an aliphatic-hydroxyl amino acid at position 118. In another aspect, the VHPO comprises an amino sequence having a non-polar amino acid at position 118.

In one aspect, the VHPO comprises an amino sequence having an aliphatic amino acid at position 138. In one aspect, the aliphatic amino acid at position 138 is glycine. In another aspect, the aliphatic amino acid at position 138 is alanine.

In one aspect, the VHPO comprises an amino sequence having an aliphatic-hydroxyl amino acid at position 260. In another aspect, the VHPO comprises an amino sequence having a non-polar amino acid at position 260.

In one aspect, VHPO comprises an amino sequence having an aliphatic-hydroxyl amino acid at position 536. In one aspect, the aliphatic-hydroxyl amino acid at position 536 is threonine. In one aspect, the aliphatic-hydroxyl amino acid at position 536 is serine.

In one aspect, VHPO comprises an amino sequence having a basic amino acid at position 569. In one aspect, the basic amino acid at position 569 is lysine. In another aspect, the basic amino acid at position 569 is arginine.

In one aspect, VHPO comprises an amino sequence having an aliphatic amino acid at position 588. In one aspect, the aliphatic amino acid at position 588 is valine. In another aspect, the aliphatic amino acid at position 588 is isoleucine.

In another aspect, the VHPO comprises an amino sequence having a non-polar amino acid at position 592. In one aspect, the non-polar amino acid at position 592 is proline. In another aspect, the non-polar amino acid at position 592 is leucine.

In one aspect, the VHPO comprises an amino sequence having a basic amino acid at position 594. In another aspect, the VHPO comprises an amino sequence having an acidic amino acid at position 594. In another aspect, the VHPO comprises an amino sequence having an uncharged polar amino acid at position 594.

In another aspect, the VHPO comprises an amino sequence having a non-polar amino acid at position 606.

In certain aspects, the VHPO or variant protein thereof demonstrates improved properties compared to a reference VHPO. The improved properties of the VHPO or variant protein thereof include increased substrate specificity, substrate selectivity, substrate binding, cofactor specificity, cofactor selectivity, cofactor binding, enzyme activity, enzyme kinetics, enzyme expression, enzyme stability, enzyme biocatalysis (i.e., titer, rate, yield, specific yield, and total turnover number), enzyme glycosylation, enzyme methylation, enzyme acetylation, retention of the enzyme in the cell or sub-cellular component, secretion of the enzyme from the cell, and/or enzyme activity across a broader temperature range or pH range compared to the reference VHPO. In one aspect, the reference VHPO comprises SEQ ID NO: 1. In another aspect, the reference VHPO comprises SEQ ID NO: 2.

TABLE 1
VHPO amino acid sequences of fungal strains and consensus sequence.

| | SEQ | |
|---|---|---|
| Fungal | ID | |
| Strain | NO: | VHPO Amino Acid Sequence |
| Curvularia | 1 | MGSVTPIPLPKIDEPEEYNTNYILFWNHVGLELNRVTHTVGGPLTGPPLSARALGMLHLAIHDAYFSICPPTDF |
| inaequalis | | TTFLSPDTENAAYRLPSPNGANDARQAVAGAALKMLSSLYMKPVEQPNPNPGANISDNAYAQLGLVLDRSV |
| PDB ID | | LEAPGGVDRESASFMFGEDVADVFFALLNDPRGASQEGYHPTPGRYKFDDEPTHPVVLIPVDPNNPNGPKMP |
| 1VNC* | | FRQYHAPFYGKTTKRFATQSEHFLADPPGLRSNADETAEYDDAVRVAIAMGGAQALNSTKRSPWQTAQGLY |
| | | WAYDGSNLIGTPPRFYNQIVRRIAVTYKKEEDLANSEVNNADFARLFALVDVACTDAGIFSWKEKWEFEFWR |
| | | PLSGVRDDGRPDHGDPFWLTLGAPATNTNDIPFKPPFPAYPSGHATFGGAVFQMVRRYYNGRVGTWKDDEP |
| | | DNIAIDMMISEELNGVNRDLRQPYDPTAPIEDQPGIVRTRIVRHFDSAWELMFENAISRIFLGVHWRFDAAAA |
| | | RDILIPTTTKDVYAVDNNGATVFQNVEDIRYTTRGTREDEEGLFPIGGVPLGIEIADEIFNNGLKPTPPEIQPMP |
| | | QETPVQKPVGQQPVKGMWEEEQAPVVKEAP |
| Curvularia | 2 | MGSITPIPLPKIDEPEEYNTNYILFWNHVGLELNRVTHTVGGPLTGPPLSARALGMLHLAIHDAYFSIYPPTDFS |
| sp. 4388 | | TFLSPNAENAAYRLPSPNGANDARQAVAGAALKMLTSLYMKPVETPNPNPGANISDNAYAQLALVIDRSVL |
| | | KAPGGVDRESASFMFGETVADVFFALLNDPRGASQEGYHPTPGRYKFDDEPTHPVVLIPVDPNNPNGPKKPF |
| | | RQYHAPFYGKTTKRFATQSEHFLADPPGLRSNADETAEYDDAIRVAIAMGGAQALNSTKRSPWQTAQGLFW |
| | | AYDGSNLIGTPPRFYNQIVRRIAVTYKKEEDLANSEVNNADFARLFALVDVACTDAGIFSWKEKWEYEFWRP |
| | | LSGVRDDGRPDHGDPFWLTLGAPATNTNDIPFKPPFPAYPSGHATFGGAVFQMVRRYYNGRVGTWKDDEPD |
| | | NIAIDMMISEELNGLNRDLRQPYDPTAPIEDQPGIVRTRIVRHFDSAWELMFENAISRIFLGVHWRFDAAAARD |
| | | ILIPTTTKDVYAVDNNGATVFQNVEDIRYTTKGTREDREGLFPIGGVPLGIEIADEIFNNGLKPTPPELQPMPQQ |
| | | TPVQKPVGQQPVQGMWAEEQAPVIKEAP |
| Curvularia | 3 | MGSVTPIPIPRIDEPEEYNTNYILFWNHVGLELNRITHTVGGPLTGPPLSARALGMLHLAIHDAYFSICPPSDFTT |
| specifera | | FLSPDAENVAYRLPSLNGADDARQAVAGAALKMLSLLYMKPVEQPNPNPGANISDNAYAQLGLVLDRSVLK |
| US-188 | | APGGVDQESASFMFGEAVADVFFALLNDPRGASQEGYHPTLGRYKFDDEPTHPVVLIPVDPNNPNGPKKPFR |
| | | QYHAPFYGKTTKRFATQSEHFLADPPGLRSNADETAEYDDSIRVAIAMGGAQGLNSTKRSPWQTAQGLYWA |
| | | YDGSNLIGTPPRFYNQIVRRIAVTYKKEEDLANSEVNNADFARLFALVNVACTDAGIFSWKEKWEFEFWRPL |
| | | SGVRDDGRPDHGDPFWLTLGAPATNTNDIPFKPPFPAYPSGHATFGGAVFQMVRRYYNGRVGTWKDDEPDN |
| | | IAIDMMISEELNGLNRDLRQPYDPTAPIEDQPGIVRTRIVRHFDSAWEMMFENAISRIFLGVHWRFDAAAARDI |
| | | LIPTTTKDVYAVDNNGATVFQNVEDVRYSTKGTREGREGLFPIGGVPLGIEIADEIFNNGLRPTPPELQPMPQE |
| | | TPVQKPVEGMWEEEQAPIVNEAP |
| Curvularia | 4 | MAAITPIPLPKIDEPEEYNTNYILYWNHVGLELNRVTHTVGGPLTGPPLSARALGMLHLAIHDAYFSIHPSADF |
| intermedia | | HTFLSPDATDPAYRLPTPQGANDARQAVAGASLKMLTSLYSKPTVQPNPNPGANISDNAYAQLGLVLDRSAL |
| US-429 | | KAPGGVDRASASFSFGEAVADVFFALLNDPRGASQEGYHPTPGRYKFDDEPTHPVTLIPVDPNNPNGPKKPFR |
| | | QYHAPFYGKTTKRLATQTEHMLADPPGLRSNANETAEYDDAVRVAIAMGGAQGLNSTKRSPWQTVQGMY |
| | | WAYDGSNLIGTPPRFYNQIVRRIAVTYKKEADLANSEVNNADFARLFALVDVACADAGIFSWKEKWEFEFW |
| | | RPLSGVRDDGRPDHADPFWLTLGAPSTNTNDIPFKPPFPAYPSGHATFGGAVFQMVRRYYNGRVGAWKNDE |
| | | IDNIAIDMMVSEELNGLNRDLRQPYDPTAPIEDQPGIVRTRITRHFGSAWELMFENAISRIFLGVHWRFDAAAA |
| | | RDILIPTTTKDVYAVDGTGATVFQNIEDVKYTTRGTRQGCEGLYPIGGVPLGMEIANEIFESGLRPTPAERQPK |
| | | VKEADMQETVGGMWEGEEAPLVNEAP |
| Curvularia | 5 | MGSVIPIPLPKIDEPEEYNTNYVLFWNNVGLELNRVTHTVGGPLTGPPLSARALGMLHLAIHDAYFSIYPPTDF |
| pseudobra- | | TTFLSPDAENAAYRLPSLNGANDARQAVAGAALKMLSSLYMKPDSSSGGSISDNAYAQLALVIDRSVLKAPG |
| chyspora | | GVDRESASFVFGEAVADVFFALLNDPRGASQEGYHPTPGRYKFDDEPTHPVVLVPVDPNNPNGPKKPFRQYH |
| US-215 | | APFYGKTTKRFATQTEHFLADPPGLRSNADETAEYDDSIRVAVAMGGAQGLNSTKRSPWQTAQGLFWAYDG |
| | | SNLVGTPPRFYNQIVRRIAVTYKKEEDLANSEVNNADFARLFALVDVACTDAGIFSWKEKWEYEFWRPLSGV |
| | | RDDGRPDHGDPFWLTLGAPATNTNDIPFKPPFPAYPSGHATFGGAVFQMVRRYYNGRVGTWKDDEPDNIAI |
| | | DMVISEELNGLNRDLRQPYDPTAPIEDQPGIVRTRIVRHFDSAWEMMFENAISRIFLGVHWRFDAAAARDILIP |
| | | TTTKDVYAVDNNGATVFQNVEDIRYSTKGTREDREGLFPIGGVPLGIEIADEIFNNGLRPTPPELQPMPQETPV |
| | | QKPIGQQLVQGLWHDEEGQVPVVKEAP |
| Curvularia | 6 | MGSVTPIPLPRIDEPEEYNTNYILFWNNAGLELNRVTHTVGGPLTGPPLSARALGMLHLAIHDAYFSIFPPSDFT |
| hawaiiensis | | TFLSPDAENAAYRLPSLNGADDARQAVAGAALKMLSLLYMKPIEQPNPNPGANISDNAYAQLGLVLDRSAL |
| US-446 | | KAPGGVDRESVSFMFGEAVADVFFALLNDPRGASQEGYHPTPGRYKFDDEPTHPVVLIPVDPNNPSGPKMPF |
| | | RQYHAPFYGKTTKRFATQSEHFLADPPGLRSNADETAEYDDSIRTAIAMGGAPGLNSTKRSPWQTAQGLYW |
| | | AYDGSNLIGTPPRFYNQIVRRIAVTYKKEEDLANSEVNNADFARLFALVDVACTDAGIFSWKEKWEFEFWRP |
| | | LSGVRDDGRPDHGDPTWLTLGAPATNTNDIPFKPPFPAYPSGHATFGGAVFQMVRRYYNGRVGTWKDDEPD |
| | | NIAIDMMISEELNGLNRDLRQPYDPTAPIEDQPGIVRTRIVRHFDSAWELMFENAISRIFLGVHWRFDAAAARD |
| | | ILIPTTTKDVYAVDNNGATIFQNVEDIRYSTKGTREDREGLFPIGGVPLGIEIADEIFNNGLKPTPPELQPMPQET |
| | | PVQKPVGQQPVKGMWEEEQAPIVKEAP |
| Curvularia | 7 | MGSVTPIPLPKIDEPEEYNTNYILFWNYVGLELNRVTHTVGGPLTGPPLSARALGMLHLAVHDAYFSICPPTDF |
| protuberata | | TTFLSPDAENAAYRLPSTNGANDARQAVAGAALKMLTSLYMKPIEQPNPNPSANISDNAYAQLALVLDRSA |
| US-1111 | | MEAPGGIDRESASFMFGEAVADVFFALLNDPRGASQEGYHPTPGRYKFDDEPTHPVVLIPVDPNNPNGPKKPF |
| | | RQYHAPFYGKTTKRFATQSEHFLADPPGLRSNADETAEYDDAIRVAIAMGGAQALNSTKRSPWQTAQGLYW |
| | | AYDGSNLIGTPPRFYNQIVRRIAVTYKKEEDIANSEVNNADFARLFALVDVACTDAGIFSWKEKWEFEFWRPL |
| | | SGVRDDGRPDHGDPFWLTLGAPATNTNDIPFKPPFPAYPSGHATFGGAVFQMVRRYYNGRVGTWKDDEPDN |
| | | IAIDMMISEELNGLNRDLRQPYDPTAPIEDQPGIVRTRIVRHFDSAWELMFENAISRIFLGVHWRFDAAAARDI |
| | | LIPTTTKDVYAVDNNGATVFQNVEDIRYTTKGTREDREGLFPIGGVPLGIEIANEIFENGLKPTPPEIQPMPQET |
| | | PAQKPVKGMWQEEQVPVVEEAP |
| Curvularia | 8 | MGSVIPIPLPKIDEPEEYNTNYVLFWNNVGLELNRVTHTVGGPLTGPPLSARALGMLHLAIHDAYFSIYPPTDF |
| lunata | | TTFLSPDAENAAYRLPSLNGANDARQAVAGAALKMLSSLYMKPDSSSGGSISDNAYAQLALVIDRSVLKAPG |
| US-991 | | GVDRESASFVFGEAVADVFFALLNDPRGASQEGYHPTPGRYKFDDEPTHPVVLVPVDPNNPNGPKKPFRQYH |
| | | APFYGKTTKRFATQTEHFLADPPGLRSNADETAEYDDSIRVAVAMGGAQGLNSTKRSPWQTAQGLFWAYDG |
| | | SNLVGTPPRFYNQIVRRIAVTYKKEEDLANSEVNNADFARLFALVDVACTDAGIFSWKEKWEYEFWRPLSGV |
| | | RDDGRPDHGDPFWLTLGAPATNTNDIPFKPPFPAYPSGHATFGGAVFQMVRRYYNGRVGTWKDDEPDNIAI |
| | | DMVISEELNGLNRDLRQPYDPTAPIEDQPGIVRTRIVRHFDSAWEMMFENAISRIFLGVHWRFDAAAARDILIP |
| | | TTTKDVYAVDNNGATVFQNVEDIRYSTKGTREDREGLFPIGGVPLGIEIADEIFNNGLRPTPPELQPMPQETPV |
| | | QKPIGQQLVQGLWHDEEGQVPVVKEAP |
| Consensus | 9 | MGSVTPIPLPKIDEPEEYNTNYILFWNHVGLELNRVTHTVGGPLTGPPLSARALGMLHLAIHDAYFSIXPPTDF |
| Sequence | | TTFLSPDAENAAYRLPSLNGANDARQAVAGAALKMLSSLYMKPVEQPNPNPGANISDNAYAQLXLVLDRSV |
| | | LKAPGGVDRESASFMFGEAVADVFFALLNDPRGASQEGYHPTPGRYKFDDEPTHPVVLIPVDPNNPNGPKKP |
| | | FRQYHAPFYGKTTKRFATQSEHFLADPPGLRSNADETAEYDDXIRVAIAMGGAQGLNSTKRSPWQTAQGLY |
| | | WAYDGSNLIGTPPRFYNQIVRRIAVTYKKEEDLANSEVNNADFARLFALVDVACTDAGIFSWKEKWEFEFWR |
| | | PLSGVRDDGRPDHGDPFWLTLGAPATNTNDIPFKPPFPAYPSGHATFGGAVFQMVRRYYNGRVGTWKDDEP |
| | | DNIAIDMMISEELNGLNRDLRQPYDPTAPIEDQPGIVRTRIVRHFDSAWELMFENAISRIFLGVHWRFDAAAAR |
| | | DILIPTTTKDVYAVDNNGATVFQNVEDIRYXTKGTREDREGLFPIGGVPLGIEIADEIFNNGLXPTPPELQPMPQ |
| | | ETPVQKPXGQQXVXGMWEEEQAPVVKEAP |

*Messerschmidt A, Wever R. X-ray structure of a vanadium-containing enzyme: chloroperoxidase from the fungus Curvularia inaequalis. Proc Natl Acad Sci U.S.A. 1996 Jan. 9;93(1): 392-6. doi: 10.1073/pnas.93.1.392. PMID: 8552646; PMCID: PMC40244.

### Deposited Fungal Strains

Biological deposits of each of the fungal strains listed in Table 2 were made on the dates shown at the American Type Culture Collection (ATCC®), located at 10801 University Blvd., Manassas, VA 20110, USA, or the National Measurement Institute (NMI), 1/153 Bertie Street, Port Melbourne, Victoria 3207, Australia, under the provisions of the Budapest Treaty and assigned by each International Depositary Authority (IDA) the accession numbers indicated. Upon issuance of a patent, all restrictions upon the deposits will be irrevocably removed. The deposits are intended to meet the requirements of 37 CFR §§ 1.801-1.809. The deposits will be maintained in the IDAs for a period of 30 years, or 5 years after the last request, or for the effective, enforceable life of the patent, whichever is longer, and will be replaced, if necessary, during that period; and the requirements of 37 CFR §§ 1.801-1.809 are met.

TABLE 2
Fungal strains deposited with the ATCC and NMI International Depositary Authorities.

| Strain | | | | Accession |
|---|---|---|---|---|
| Number | Species | IDA | Date of Deposit | No. |
| 4388 | Curvularia sp. | NMI | 10 Jun. 2022 | V22/011149 |
| US-991 | Curvularia lunata | ATCC | 18 Jan. 2023 | PTA-127504 |
| US-215 | Curvularia | ATCC | 24 Jan. 2023 | PTA-127509 |
| | pseudobrachyspora | | | |
| US-188 | Curvularia | ATCC | 24 Jan. 2023 | PTA-127510 |
| | spicifera | | | |
| US-998 | Curvularia | ATCC | 24 Jan. 2023 | PTA-127511 |
| | inaequalis | | | |
| US-429 | Curvularia | ATCC | 24 Jan. 2023 | PTA-127512 |
| | intermedia | | | |
| US-446 | Curvularia | ATCC | 16 May 2023 | PTA-127601 |
| | hawaiiensis | | | |
| US-1111 | Curvularia | ATCC | 13 Jun. 2023 | PTA-127607 |
| | protuberata | | | |

### Animal Feed

Also disclosed herein is an animal feed comprising the compositions described herein (i.e., a composition comprising a fungal strain, biomass from the fungal strain, a culture supernatant from the fungal strain, or a combination thereof). The animal feed may be solid (e.g., powder, granules, pellets, feed block, lick block), semi-solid (e.g., gel, ointment, cream, paste) or liquid (e.g., solutions, suspensions, emulsions). The animal feed may independently be solid, semi-solid (e.g., gel, ointment, cream, paste) or liquid (e.g., solutions, suspensions, emulsions). For example, the animal feed may both be liquid or both be semi-solid or both be solid. Alternatively, the animal feed and composition may each be a different physical state. For example, the animal feed may be solid or semi-solid and the composition may be liquid. The composition may, for example, be used to “top-dress” (added on top) a ruminant feedlot ration or may be used to blend into a total mixed ration. In one aspect, the animal feed is in the form of a feed block. In another aspect, the animal feed is in the form of a lick block. In another aspect, the animal feed is in the form of a low moisture block.

The composition may, for example, be added to the drinking water of the animal. In certain embodiments, the composition may be added to the drinking water of the animal immediately before ingestion, for example up to 1 hour before ingestion or up to 30 minutes before ingestion or up to 15 minutes before ingestion or up to 5 minutes before ingestion.

The three main types of animal feed include roughages, concentrates and mixed feeds. In general, roughages contain a higher percentage of crude fiber and a lower percentage of digestible nutrients than concentrates. For example, roughages may be defined as containing equal to or greater than 20 wt % crude fiber and equal to or less than 60 wt % total digestible nutrients. Roughages may include, for example, dry roughages (e.g., hay, straw, artificially dehydrated forages containing at least 90 wt % dry matter), silages (formed from green forages such as grass, alfalfa, sorghum and corn and preserved in a silo at dry matter contents of 20 to 50%), and pastures (e.g., green growing pastures providing forage that has a high water content and generally less than 30% dry matter). The two basic types of roughages include grasses and legumes. Grasses are generally higher in fiber and dry matter than legumes. Legumes are generally higher in proteins, metabolizable energy, vitamins and minerals. Concentrates contain a relatively lower percentage of crude fiber and a higher percentage of digestible nutrients than roughages. For example, concentrates may be defined as containing less than 20 wt % crude fiber and greater than 60 wt % total digestible nutrients. Concentrates may include, for example, energy-rich grains and molasses. Corn, wheat, oats, barley and milo (sorghum grain) are energy-rich grains, containing about 70 to 80 wt % total digestible nutrients.

Mixed feeds are generally a mixture of roughages and concentrates to provide “complete” balanced rations and may be either high or low in energy, protein or fiber. The disclosed compositions and fungal strains, for example, can be combined with animal feed in various amounts depending on the total amount of fungal strain, culture supernatant from the fungal strain, or combination thereof intended to be administered to the animal.

The animal feed may, for example, comprise from about 0.0001 wt % to about 10 wt % of disclosed compositions, based on the total dry weight of the animal feed. The animal feed may, for example, comprise from about 0.01 wt % to about 10 wt % of disclosed composition, based on the total dry weight of the animal feed. For example, the animal feed may comprise from about 0.001 wt % to about 9.5 wt %, or from about 0.005 wt % to about 9 wt %, or from about 0.01 wt % to about 8.5 wt %, or from about 0.05 wt % to about 8 wt %, or from about 0.1 wt % to about 7.5 wt %, or from about 0.9 wt % to about 7 wt %, or from about 1 wt % to about 6 wt %, or from about 1.5 wt % to about 5.5 wt %, or from about 2 wt % to about 5 wt %, or from about 2.5 wt % to about 4.5 wt %, or from about 3 wt % to about 4 wt % disclosed composition based on the total dry weight of the animal feed. For example, the animal feed may comprise from about 0.4 wt % to about 9.5 wt %, or from about 0.5 wt % to about 9 wt %, or from about 0.6 wt % to about 8.5 wt %, or from about 0.7 wt % to about 8 wt %, or from about 0.8 wt % to about 7.5 wt %, or from about 0.9 wt % to about 7 wt %, or from about 1 wt % to about 6 wt %, or from about 1.5 wt % to about 5.5 wt %, or from about 2 wt % to about 5 wt %, or from about 2.5 wt % to about 4.5 wt %, or from about 3 wt % to about 4 wt % disclosed composition based on the total dry weight of the animal feed.

In one embodiment, the fungal strain, biomass from the fungal strain, a culture supernatant from the fungal strain, or a combination thereof is administered at a dose of preferably at least 16.67, 10, 5, 3, 2, 1, 0.5, 0.25 0.125 or 0.067% of the dry matter administered to the ruminant animal. For example, if a 450 kg ruminant animal (e.g., steer) consumes 2.5% to 3% of its body weight per day of feed, then the fungal strain is administered at a dose proportional to the amount of dry matter administered to the ruminant. In the case of a 450 kg ruminant animal, and where 80% of the feed is dry matter, if the animal consumes about 2.5% of its body weight per day, then the fungal strain, biomass from the fungal strain, a culture supernatant from the fungal strain, or a combination thereof is administered at a dose of about 0.27, 0.18, 0.09, 0.045, 0.0225, 0.01125 or 0.00603 kg per day to result in a dose at least 3, 2, 1, 0.5, 0.25 0.125 or 0.067% of the dry matter administered to the ruminant animal.

Any embodiment of the invention is meant to be illustrative only and is not meant to be limiting to the invention. Therefore, it should be appreciated that various other changes and modifications can be made to any embodiment described without departing from the spirit and scope of the invention.

All headings are for the convenience of the reader and should not be used to limit the meaning of the text that follows the heading, unless so specified.

The present invention is further illustrated by the following examples that should not be construed as limiting. The contents of all references, patents, and published patent applications cited throughout this application, as well as the Figures, are incorporated herein by reference in their entirety for all purposes.

### EXAMPLES

### Example 1. Materials and Methods

### Fungal Strains and Culture Conditions

Fungal species were grown on pure potato dextrose agar (PDA) plates. 9 mm of agar and mycelia were taken and inoculated in 5 ml of potato dextrose liquid media (PD) and allowed to grow for three days. Cultures were scaled to a volume of 100 ml of PD. Fungi were then allowed to grow in the media for 4, 8 or 12 days at 30° C. and at 200 rpm. Supernatants were obtained by filtration and then centrifugation (5000 rpm). Supernatants were taken to determine VHPO activity.

### Haloperoxidase Enzyme Assay of Supernatants

The haloperoxidase assay was carried out in 96-well microtiter plates. Each well contained 100 mM sodium acetate, pH 5, 0.05 mM phenol red (also known as phenolsulfonphthalein or PSP), 50 mM sodium bromide, 0.1 mM orthovanadate, 10 mM hydrogen peroxide, and culture supernatant. The reaction was observed spectrophotometrically by measuring the absorption at 590 nm after 1 hour and 24 hours using an INFINITE® M200 NANOQUANT to read plates. The absorption peak at 590 nm belongs to the conversion of phenol red to bromophenol blue.

Determination of Haloperoxidase Activity from Solid Media

Fungal species were grown on potato dextrose agar (PDA) plates. 9 mm of agar and mycelia were taken and resuspended in 400 mM acetate buffer at pH 5. Samples were vortexed for 30 minutes. After vortexing, samples were centrifuged at 10000×g for 10 minutes. The supernatant was taken to measure haloperoxidase activity as outlined above.

### Example 2. Vanadium-Dependent Haloperoxidase (VHPO) Activity in the Supernatants of the Curvularia Strains

The fungal strains listed in Table 2 were cultured and their supernatants were assayed for haloperoxidase activity using the methods described in Example 1. A negative control without VHPO enzyme and a positive control with mU/ml purified VHPO were also assayed.

The absorbance measurements indicated that each of the fungal strains had haloperoxidase activity detectable above the negative control (see Table 3). As the absorbance measurements were not normalized, this initial experiment did not provide any detail regarding the relative haloperoxidase activities present in the fungal strains.

TABLE 3
Absorbance measurements at 590 nm of fungal
strain supernatants.

| | | Standard |
|---|---|---|
| Sample | Average | Error |
| Curvularia lunata US-991 | 0.1021 | 0.0076 |
| Curvularia spicifera US-188 | 0.2128 | 0.0255 |
| Curvularia pseudobrachyspora US-215 | 0.0878 | 0.0007 |
| Curvularia hawaiiensis US-446 | 0.0951 | 0.0018 |
| Curvularia intermedia US-429 | 0.0947 | 0.0040 |
| Curvularia inaequalis US-998 | 0.1879 | 0.0576 |
| (+) 0 (Negative Control) | 0.0774 | 0.0024 |
| (+) 2.5 mU/ml purified VHPO | 2.1888 | 0.2555 |
| (Positive Control) | | |

### Example 3. Structural Analysis of VHPO Proteins Expressed in the Curvularia Strains

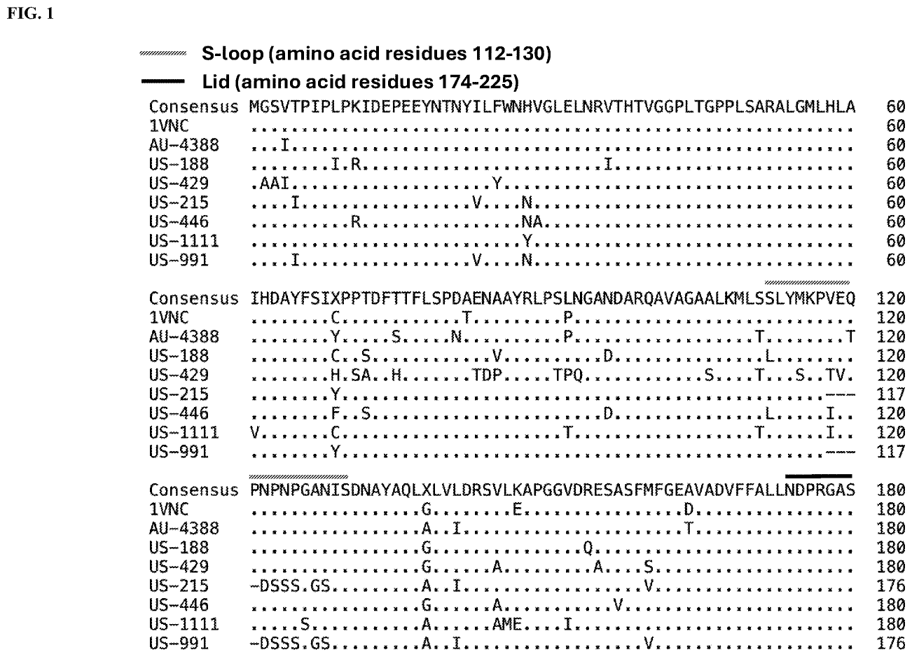

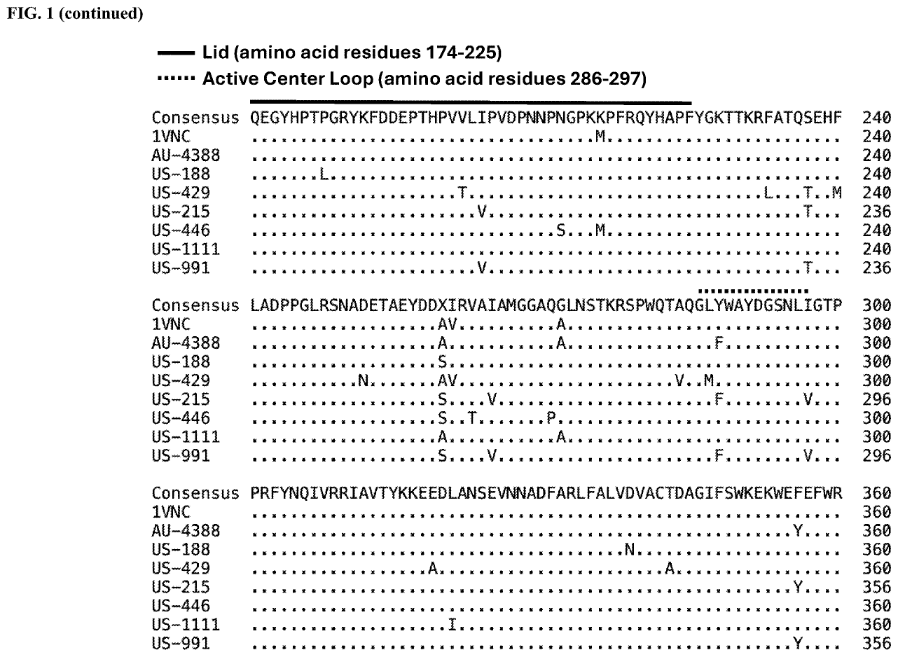

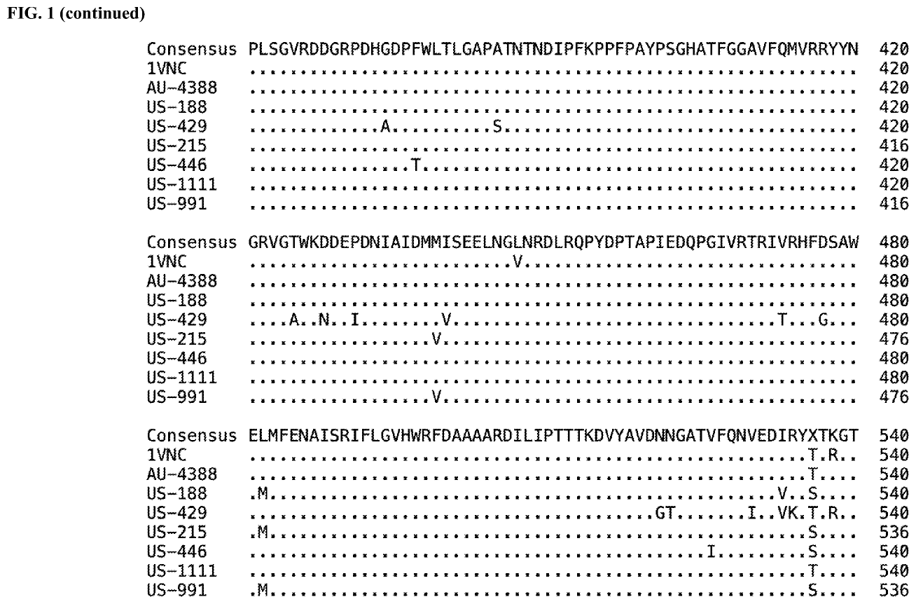

The VHPO sequences from the fungal strains Curvularia lunata US-991 (ATCC Accession No. PTA-127504), Curvularia pseudobrachyspora US-215 (ATCC Accession No. PTA-127509), Curvularia spicifera US-188 (ATCC Accession No. PTA-127510), Curvularia inaequalis US-998 (ATCC Accession No. PTA-127511), Curvularia intermedia US-429 (ATCC Accession No. PTA-127512), Curvularia hawaiiensis US-446 (ATCC Accession No. PTA-127601), Curvularia protuberata US-1111 (ATCC Accession No. PTA-127607), Curvularia inaequalis PDB ID 1VNC, and Curvularia sp. 4388 (NMI Accession No. V22/011149) were analyzed with MUltiple Sequence Comparison by Log-Expectation (MUSCLE) in Geneious Prime. FIG. 1 presents the resulting multiple sequence alignment with the consensus sequence shown as the top sequence and amino acid residues unique to each VHPO identified by their one-letter abbreviation.

The multiple sequence alignment indicates that there is conservation of the amino acids involved in the covalent binding of vanadate with the various VHPO proteins. The following amino acids are known to be involved in vanadate binding in VHPO from studies in Curvularia inaequalis: histidine 496, arginine 360, arginine 490, serine 402 and histidine 404. Each of these conserved residues is present in the VHPO amino acid sequences from Curvularia lunata US-991 (ATCC Accession No. PTA-127504), Curvularia pseudobrachyspora US-215 (ATCC Accession No. PTA-127509), Curvularia spicifera US-188 (ATCC Accession No. PTA-127510), Curvularia inaequalis US-998 (ATCC Accession No. PTA-127511), Curvularia intermedia US-429 (ATCC Accession No. PTA-127512), Curvularia hawaiiensis US-446 (ATCC Accession No. PTA-127601), and Curvularia protuberata US-1111 (ATCC Accession No. PTA-127607) (see FIG. 1).

The presence of surface exposed aromatic residues that are thought to be involved in the binding of bromide to VHPO was also observed in the multiple sequence alignment. Bromination of surface exposed aromatic residues in VHPO is one of the mechanisms proposed to enhance haloperoxidase activity. Previously, we proposed that tyrosine 69, phenylalanine 288, and tyrosine 356 play a role in bromination processes, thereby increasing VHPO activity (see WO 2023/009899). The multiple sequence alignment of protein sequences revealed that tyrosine 69, tyrosine 356, and phenylalanine 288 are present in the VHPOs expressed by Curvularia pseudobrachyspora US-215 (ATCC Accession No. PTA-127509) and Curvularia sp. 4388 (NMI Accession No. V22/011149).

A consensus sequence was generated from the multiple sequence alignment (see SEQ ID NO: 9 in Table 1). The consensus sequence contains divergent amino acid residues at the following positions (indicated as “X” in the alignment): position 69, position 138, position 260, position 536, position 569, position 588, position 592, and position 594 (see FIG. 1).

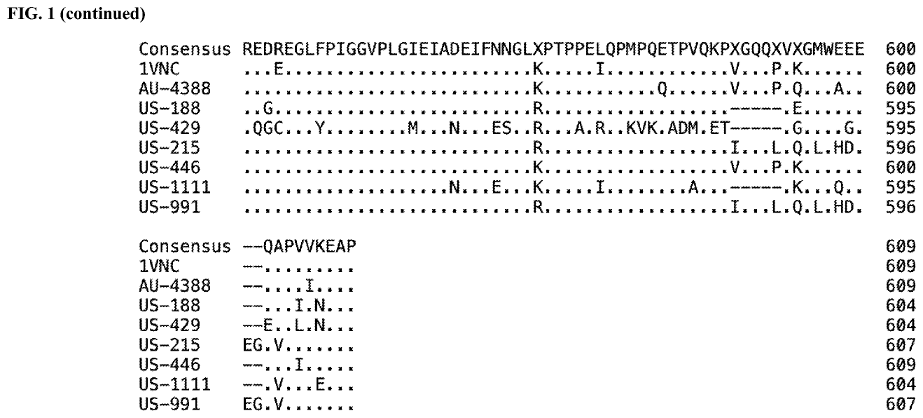

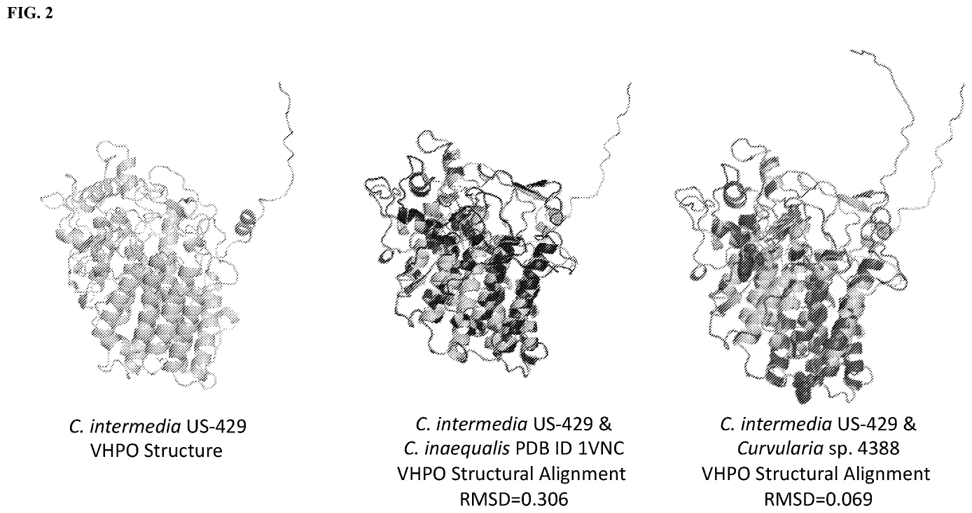

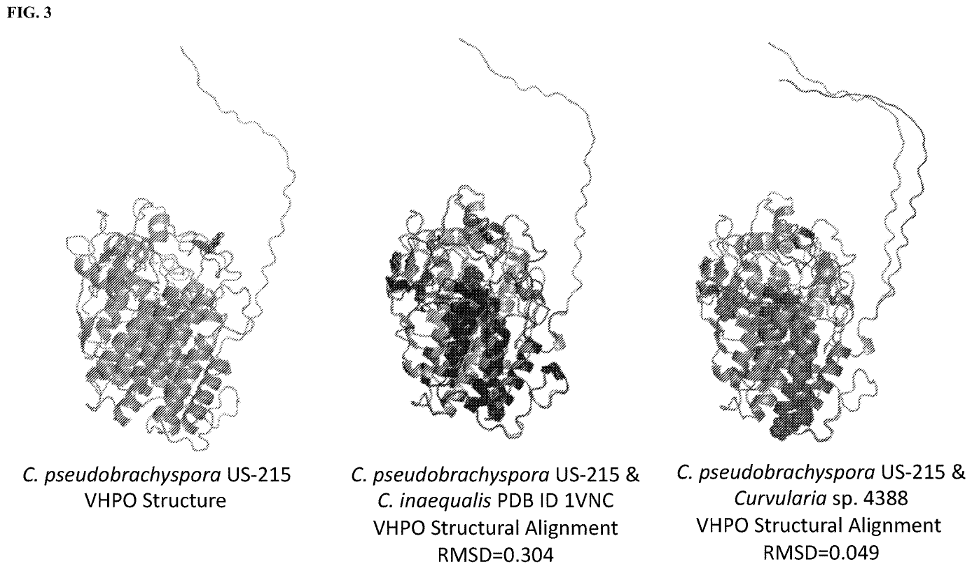

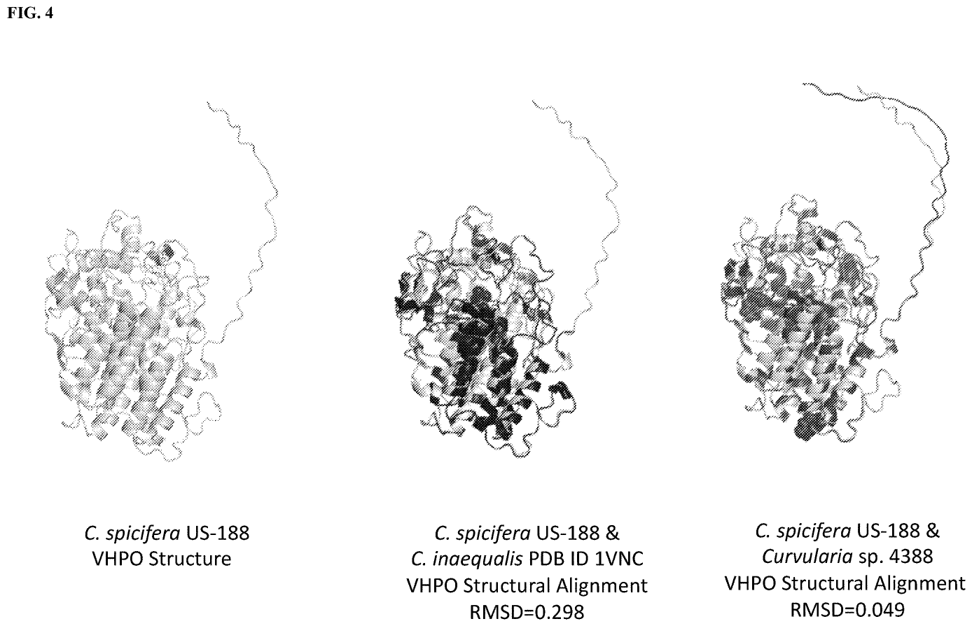

Alphafold was used to predict protein structures for VHPO enzymes expressed by Curvularia intermedia US-429 (ATCC Accession No. PTA-127512) (FIG. 2), Curvularia pseudobrachyspora US-215 (ATCC Accession No. PTA-127509) (FIG. 3), and Curvularia spicifera US-188 (ATCC Accession No. PTA-127510) (FIG. 4) (Jumper 2021). These predicted structures were subsequently aligned with the VHPO protein structures determined for Curvularia inaequalis PDB ID 1VNC, and Curvularia sp. 4388 (NMI Accession No. V22/011149). The root-mean-square deviation (RMSD), a measure of the average distance between the atoms of the superimposed protein structures, was calculated and is shown in FIGS. 2-4. The structural analysis of VHPO proteins reveals the structural conservation of the vanadate binding domain across VHPO sequences. However, it also highlights VHPO protein divergence including at bromination sites, which may be correlated with enhanced VHPO activity.

### Example 4. Computational Analysis of VHPO Proteins Expressed in the Curvularia Strains

### Definitions

“Docking pose”: predicted position and orientation of a ligand or substrate when it is bound to a protein receptor or enzyme.

“Homology model”: the prediction of the tertiary structure of an unknown protein using a known three-dimensional (3D) structure.

“Lid”: beta-sheet loop motif (i.e., residues 174-225 in FIG. 1) close to the substrate binding pocket of the VHPO.

“Loop”: any amino acid subsequence within a protein that is not of the geometrically regular type of an α-helix or a β-strand.

“Protein conformation”: the arrangement in space of the protein constituent atoms which determine the overall shape of the molecule.

“Root-mean-square fluctuations (RMSF)”: fluctuation of an atom or a group of atoms along the course of a simulation.

“S-loop”: flexible region with high sequence variation within a considered VHPO.

### Materials and Methods

### Quantum Mechanics

A ground state representation of all the studied substrates (dimedone, acetylacetone, methyl pyruvate, oxaloacetic acid, acetoacetic acid, dimethyl 1,3-acetonedicarboxylate, methyl acetoacetate, malonic acid, malic acid, citric acid, 3,3-dimethyl-1,5-cyclohexanediol) were obtained with quantum mechanics. The variational principle states that a system has never an energy value lower than the true ground state energy. Therefore, to obtain an accurate ground state representation of a system, the initial trial wavefunction should change until the expectation energy value resembles that most similar to the ground state energy. To achieve that, the compounds were geometry optimized and then point charges were calculated resorting to the RESP methodology.

### Modelling

Homology models using SEQ ID NO: 2, SEQ ID NO: 3, SEQ ID NO: 4, SEQ ID NO: 5, SEQ ID NO: 6 and SEQ ID NO: 7 (see Table 1) were built based on crystal structure 1IDQ 1 (Messerschmidt et al. 1997), which corresponds to SEQ ID NO: 1. The vanadium(V)-dioxo-dihydroxo coordinated with the catalytic His residue was geometry optimized with quantum mechanics and partial charges were calculated resorting to the RESP method (Bayly et al. 1993).

Force constants and equilibrium values for bond stretching, angle bending, and dihedrals were obtained from Gérard et al. 2023. HOBr was added to the model based on the interaction of Br— with vanadate in crystal 7QWI (vanadium-dependent bromoperoxidase) available from the RCSB Protein Data Bank.

### Molecular Dynamics Simulations

The accessible conformational space for each homology model (SEQ ID Nos: 1-7) was sampled with MD simulations. The simulations were performed with the amber parm99SB force field and with the GROMACS molecular dynamics software package. One initial energy minimization was performed, followed by two equilibration steps to slowly heat the system from 0 to 300 K. The first equilibration was performed in a canonical ensemble and the second in an isothermal-isobaric ensemble. Temperature and pressure coupling were 300 K and 1 bar, respectively, and periodic boundary conditions (PBC) were used. The particle mesh Ewald (PME) method was used to calculate electrostatic interactions. For each enzyme, three production simulations of 100 ns were carried out at 300 K in the NPT ensemble using Langevin dynamics with a collision frequency of 1.0 ps⁻¹. Constant pressure periodic boundary conditions were imposed with an average pressure of 1 atm. Isotropic position scaling was used to maintain pressure with a relaxation time of 2 ps. The time step was set to 2 fs. SHAKE constraints were applied to all bonds involving hydrogen atoms. The particle mesh Ewald (PME) method was used to calculate electrostatic interactions with a cut-off distance of 10 Å.

### Molecular Docking

Molecular docking was performed using Autodock4.2 (Morris et al. 1998) with the Lamarckian genetic algorithm (LGA) using a grid of 40×40×40 centered on the OHBr. A total of 1000 LGA runs were carried out per system. The population was 300, the GA elitism=1, the maximum number of generations was 27000, and the maximum number of energy evaluations was 2500000. Substrate conformations were sorted according to their energy and root mean square deviations.

### BACKGROUND

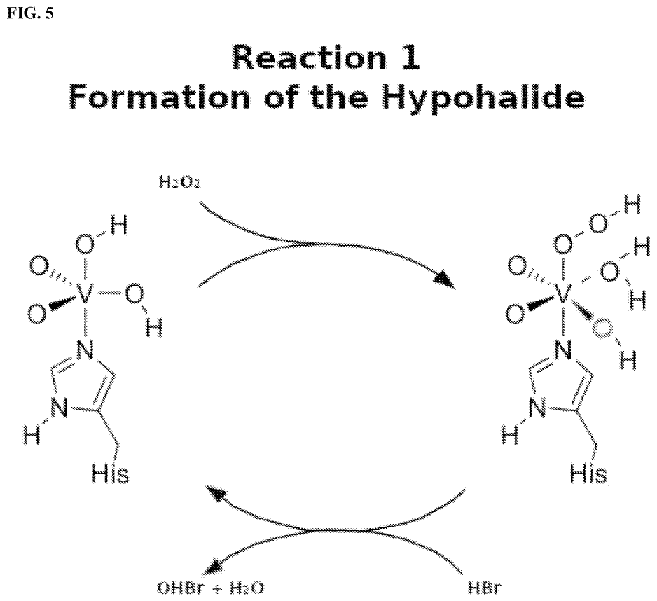

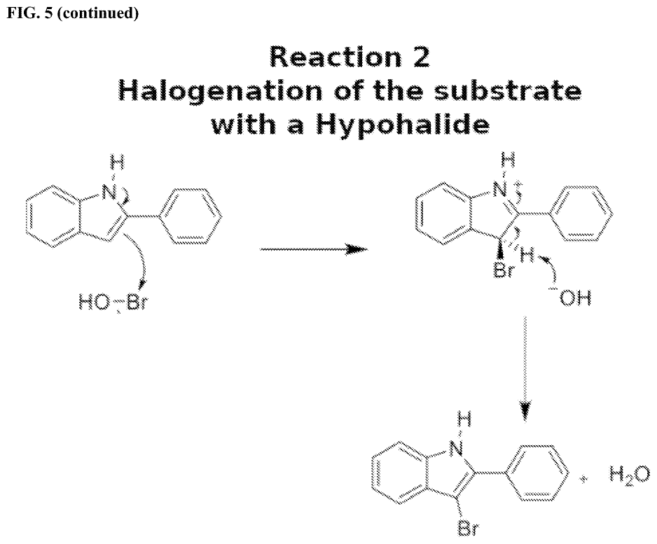

VHPO enzymes catalyze the formation of organo-halogens in the presence of H₂O₂ and halide anions (fluoride, iodide, chloride and bromide). The catalytic mechanism of VHPOs can be divided in two different reactions (FIG. 5). Reaction 1 corresponds to the formation of the hypohalide HOBr. One of hydroxy groups of the vanadium(V)-dioxo-dihydroxo abstracts a proton from a hydrogen peroxide molecule, which then nucleophilic attacks the vanadium (V) forming a vanadium(V)-dioxo-hydroxo-hydroperoxo-water six-coordinated intermediate. Then, a water is displaced to form a five-coordinated vanadium(V)-dioxo-hydroperoxo intermediate. Finally, the hydroxy from the hydroperoxo is transferred to Br—, forming the hypohalide, HOBr, while a proton is transferred to the vanadium complex to reinstate the initial vanadium(V)-dioxo-dihydroxo species (Mubarak et al. 2020). Reaction 2 describes the formation of the halogenated compound. Despite all efforts, the full reaction mechanism has not yet been determined at the QM and QM/MM level (Gérard et al. 2023). Moreover, the substrate pocket is still unknown.

### Results

In this present work, molecular dynamics simulations and molecular docking were used to determine the substrate:enzyme Michaelis-Menten complexes. Quantum Mechanics calculations were used to optimize the geometry of vanadium(V)-dioxo-dihydroxo species coordinated with the catalytic His residue. Since the active center pocket is 100% conserved in all the enzymes under study, it was decided to run long Molecular Dynamics simulations before the docking to better sample the conformation arrangement of the active centers. The reaction coordinate associated with the nucleophilic attack of the substrate on OHBr, the first step of the reaction 2 mechanism, was monitored. To do so, the hypohalide, HOBr, was added to the models of enzymes SEQ ID NOs: 1-7 based on the interaction of Br— with vanadate in crystal 7QWI (vanadium-dependent bromoperoxidase), which was published just in 2022 in the Worldwide Protein Data Bank under PDB Entry 7QWI.

### Molecular Dynamics Simulations Analysis

As mentioned above, all the seven analyzed VHPOs have a conserved active center with the overall percentage identities varying between 85 to 96%. Accordingly, all enzymes were subjected to MD simulations to sample the accessible conformational space of these enzymes. Differences in the flexibility of some regions were observed as denoted by the root-mean square fluctuations (RMSFs) (see Table 4). A higher RMSF is indicative of greater flexibility.

TABLE 4
Residues with an average RMSF above 0.3 for each of SEQ ID NO: 1 to SEQ ID NO: 7 (excluding the N-
and C-terminal regions). Averages (AVG) and standard deviations (SD) are indicated. Cells in the table
left blank correspond to those RMSF calculations that were below 0.3.

| | | SEQ ID | | SEQ ID | | SEQ ID | | SEQ ID | | SEQ ID | | SEQ ID | | SEQ ID | |
|---|---|---|---|---|---|---|---|---|---|---|---|---|---|---|---|
| | | NO: 1 | | NO: 2 | | NO: 3 | | NO: 4 | | NO: 5 | | NO: 6 | | NO: 7 | |
| Region | Residue | AVG | SD | AVG | SD | AVG | SD | AVG | SD | AVG | SD | AVG | SD | AVG | SD |
| S-loop | 118 | | | | | | | 0.33 | 0.06 | | | | | | |
| | 119 | | | | | 0.33 | 0.1 | 0.38 | 0.07 | 0.31 | 0.02 | | | | |
| | 120 | 0.37 | 0.05 | | | | | 0.45 | 0.06 | 0.3 | 0.01 | | | 0.32 | 0.15 |
| | 121 | 0.37 | 0.09 | | | | | 0.44 | 0.07 | 0.31 | 0.05 | | | 0.31 | 0.13 |
| | 122 | 0.32 | 0.05 | | | 0.31 | 0.13 | 0.39 | 0.08 | 0.31 | 0.1 | | | | |
| | 123 | 0.35 | 0.07 | | | | | 0.4 | 0.09 | | | | | | |
| | 124 | 0.35 | 0.08 | | | | | 0.36 | 0.07 | | | | | | |
| | 126 | | | | | | | 0.33 | 0.12 | | | | | | |
| Lid | 200 | | | | | | | 0.33 | 0.07 | | | | | | |
| | 203 | | | | | | | 0.31 | 0.1 | | | | | | |
| | 204 | | | | | | | 0.38 | 0.19 | | | | | | |
| | 205 | | | 0.33 | 0.11 | | | 0.46 | 0.29 | | | 0.34 | 0.1 | | |
| | 206 | | | 0.37 | 0.13 | | | 0.52 | 0.33 | | | 0.39 | 0.09 | | |
| | 207 | 0.38 | 0.22 | 0.44 | 0.17 | | | 0.58 | 0.45 | | | 0.44 | 0.12 | 0.49 | 0.04 |
| | 208 | 0.45 | 0.27 | 0.47 | 0.19 | 0.33 | 0.24 | 0.61 | 0.49 | | | 0.5 | 0.17 | 0.53 | 0.06 |
| | 209 | 0.49 | 0.32 | 0.51 | 0.23 | 0.35 | 0.27 | 0.67 | 0.56 | | | 0.51 | 0.17 | 0.58 | 0.09 |
| | 210 | 0.46 | 0.29 | 0.51 | 0.22 | 0.34 | 0.25 | 0.67 | 0.5 | | | 0.51 | 0.14 | 0.56 | 0.07 |
| | 211 | 0.45 | 0.26 | 0.47 | 0.19 | 0.31 | 0.2 | 0.66 | 0.41 | | | 0.49 | 0.11 | 0.52 | 0.06 |
| | 212 | 0.47 | 0.24 | 0.52 | 0.21 | 0.34 | 0.22 | 0.65 | 0.39 | | | 0.52 | 0.11 | 0.52 | 0.06 |
| | 213 | 0.39 | 0.15 | 0.46 | 0.18 | 0.31 | 0.19 | 0.6 | 0.37 | | | 0.48 | 0.1 | 0.48 | 0.07 |
| | 214 | 0.34 | 0.11 | 0.45 | 0.17 | 0.31 | 0.18 | 0.53 | 0.3 | | | 0.46 | 0.09 | 0.43 | 0.05 |
| | 215 | | | | | | | 0.43 | 0.21 | | | 0.37 | 0.07 | 0.34 | 0.06 |
| | 216 | | | | | | | 0.37 | 0.17 | | | 0.32 | 0.05 | | |
| | 368 | | | | | | | | | 0.33 | 0.06 | | | | |
| | 372 | | | | | | | | | | | 0.3 | 0.14 | | |

SEQ ID NO: 2 expressed in Curvularia sp. 4388 (NMI Accession No. V22/011149) was selected as a point of reference for comparison of the other VHPO sequences as it had previously been described in WO 2023/009899. Thus, many of the following analyses identify differences between the various VHPO enzymes and that presented as SEQ ID NO: 2.

The regions of the enzymes with higher RMSF values are: a loop and adjacent alpha-helix spanning residues 112 to 130 (herein designated as the “S-loop” or “loop”); a large beta-sheet loop motif (residues 174-225) close to the substrate binding pocket (herein designated as the “Lid”); and a loop containing a small alpha-helix (residues 85-87) in the N-terminal region. The S-loop displays high variability in sequence across these enzymes (see FIG. 1 and Table 5).

TABLE 5
Amino acid sequence changes for each enzyme in relation
to that of SEQ ID NO: 2 within the S-loop.
Sequences Differences in Relation to SEQ ID NO: 2 in
the S-Loop

| | |
|---|---|
| SEQ ID NO: 1 | Q120 |
| SEQ ID NO: 3 | L112, Q120 |
| SEQ ID NO: 4 | T118, V119, Q120 |
| SEQ ID NO: 5 | Del 117-120, D118(122) |
| SEQ ID NO: 6 | L112, I118, Q120 |
| SEQ ID NO: 7 | S126, Q120 |

SEQ ID NO: 2 has lower RMSFs (i.e., has greater stability) for the S-loop than SEQ ID NOs: 1, 3, 4, 5 and 7. All sequences except SEQ ID NOs: 5 and 7 have higher RMSFs for the lid than does SEQ ID NO: 2.

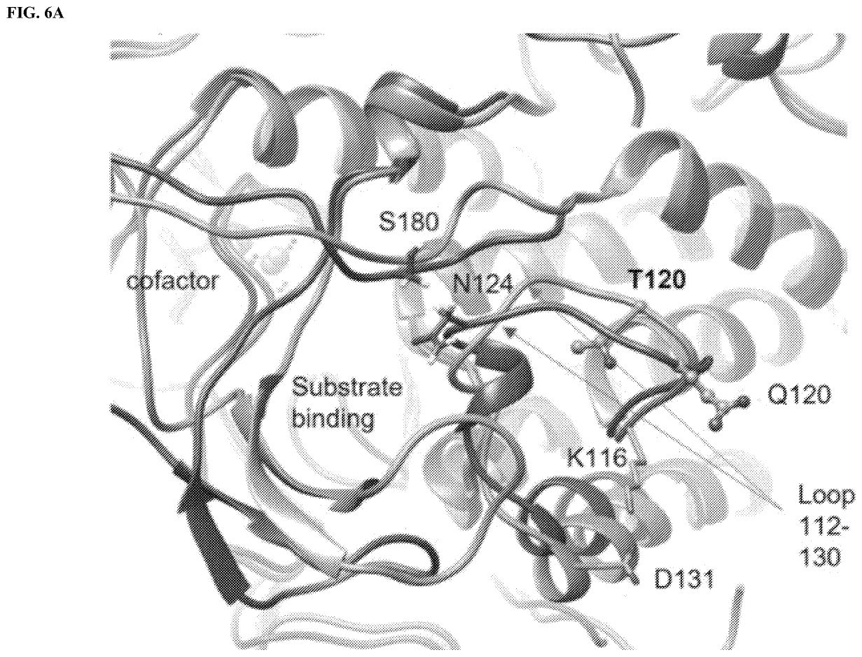

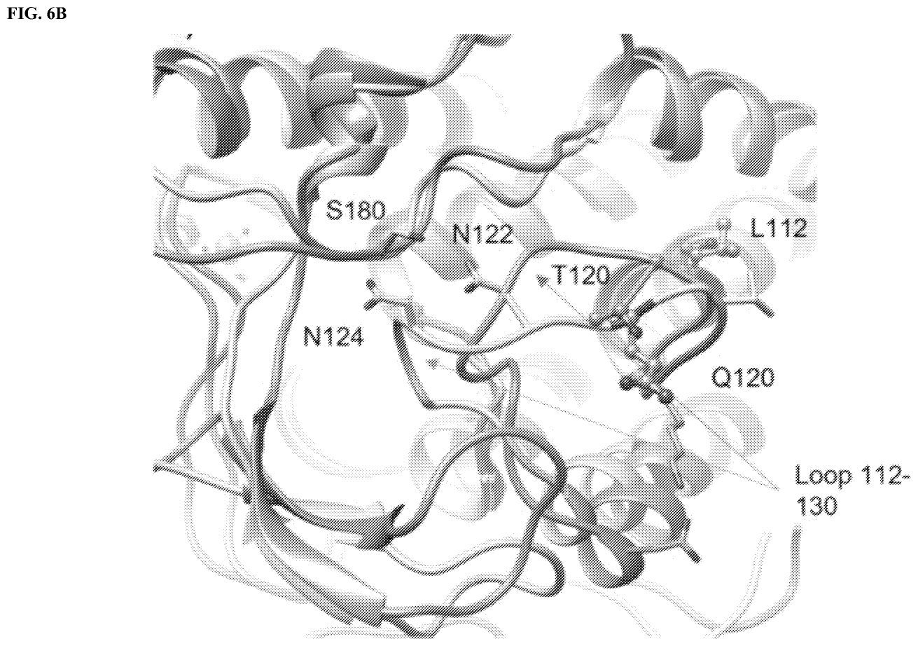

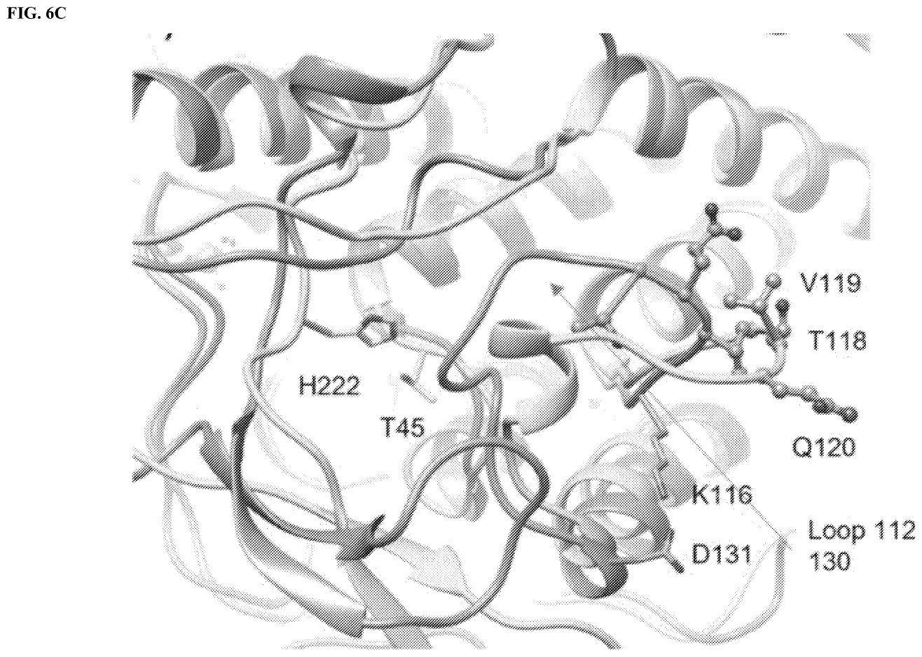

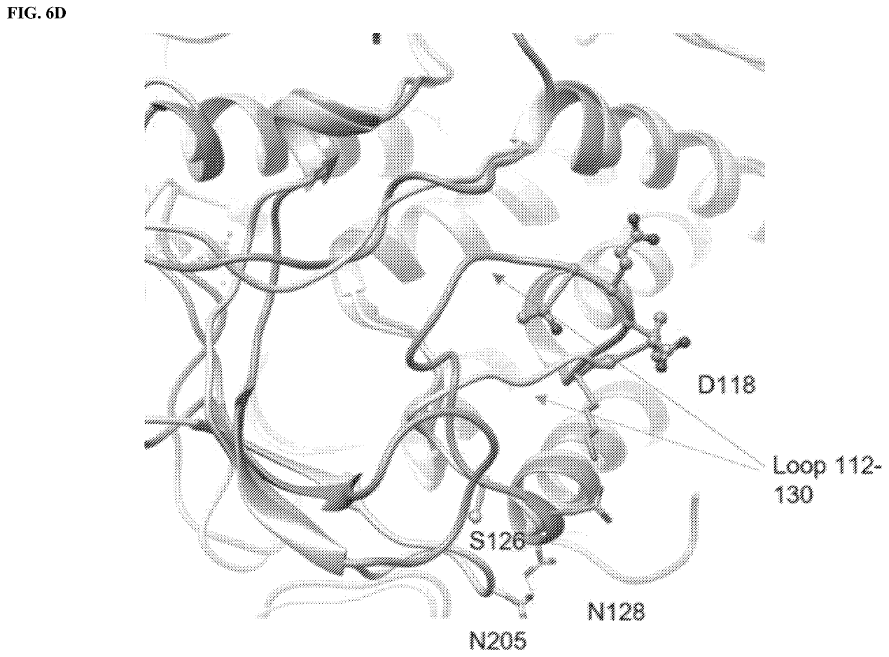

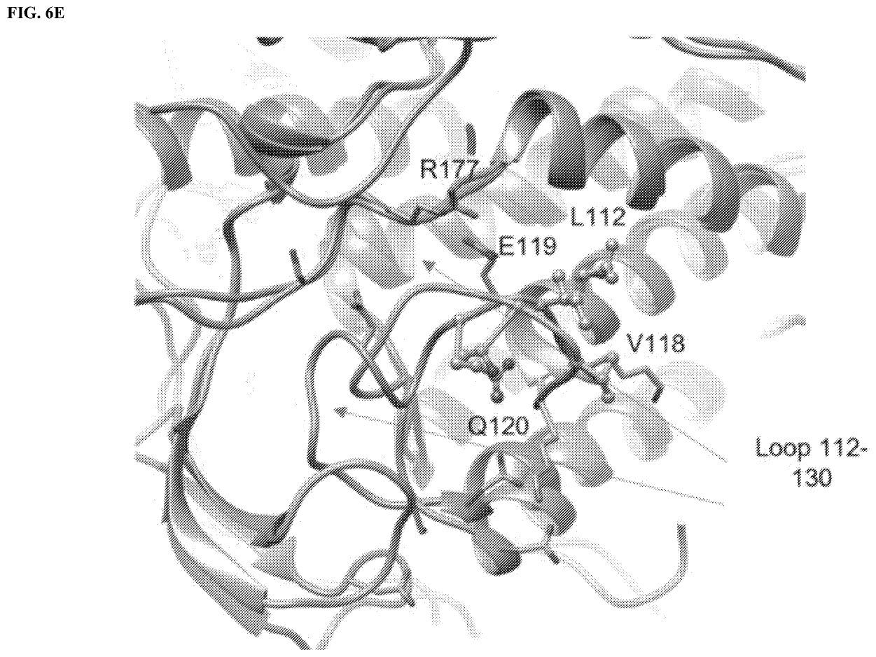

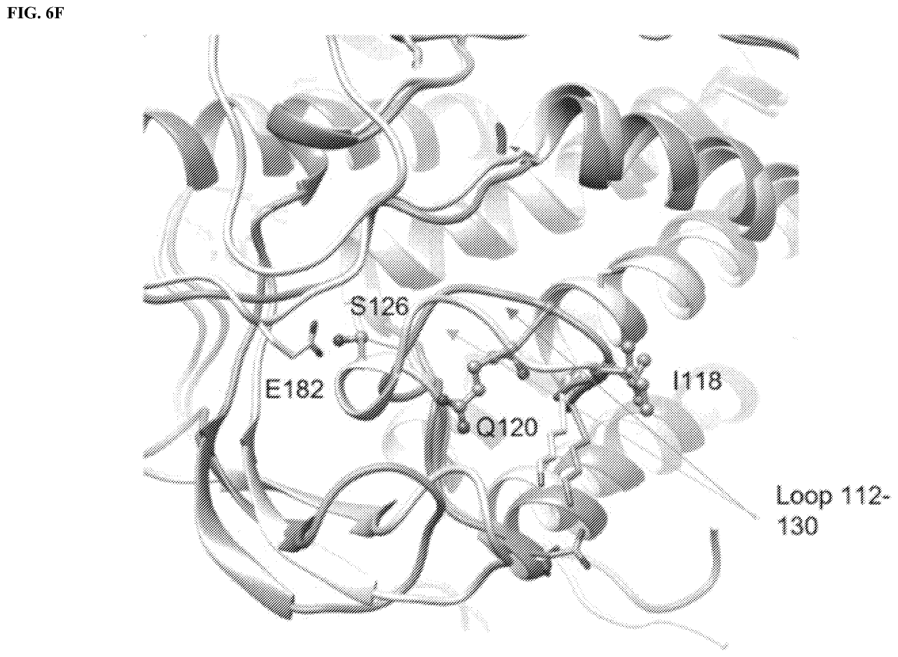

SEQ ID NO: 1 has Q120 while SEQ ID NO: 2 has a T120 (see Table 5 and FIG. 6A). This sequence difference leads to a more open loop in SEQ ID NO: 1 due to the large and more polar side chain of the glutamine residue that is solvent exposed (FIG. 6A). SEQ ID NO: 3 has the following sequence differences: Q120 and a L112 (Table 5). The S-loop in SEQ ID NO: 3 is closer to the lid (FIG. 6B), and the N124(loop):S180(lid) hydrogen bond (H-bond) is observed in the reference structure. SEQ ID NO: 4 has the following sequence differences: T118T; V119; Q120 (Table 5). The E119 in the loop of SEQ ID NO: 2 makes a long-range salt-bridge with K108. SEQ ID NO: 4 has V119 in this position, and hence it is not able to make this interaction (FIG. 6C). The lack of this interaction, and the effect of Q120 opens the S-loop in SEQ ID NO: 4 (FIG. 6C). SEQ ID NO: 5 has the deletion 117-120 in the S-loop, which makes it the smallest loop of all the analyzed sequences (Table 5, FIG. 6D). The loop can't interact with the lid as it does with the other enzymes. SEQ ID NO: 5 has a more stable lid and slightly higher loop RMSFs than SEQ ID NO: 2. SEQ ID NO: 6 has the following sequences differences: L112; I118; Q120 (Table 5). L112 and I118 interact via Van der Walls interactions, allowing the formation of a salt bridge between the E119 (loop) and R177 (lid) and a H-bond between N124(loop):S180(lid) (FIG. 6E). Accordingly, these interactions seem to counteract the effect of Q120, making SEQ ID NO: 6 more stable in the S-loop than SEQ ID NOs: 1, 3, 4, 5 and 7 (FIG. 6E). SEQ ID NO: 7 has the following sequences differences: G126S; T120 (Table 5). Its S-loop flexibility is similar to SEQ ID NO: 1 (FIG. 6F).

The differences in conformation of the S-loop for the reference structures were confirmed by following the H-bonds involving the loop, lid and substrate binding pocket during the simulations (Table 6).

All enzymes, except SEQ ID NO: 6 make an internal charged H-bond between K116 and D131. In SEQ ID NO: 6 that is replaced by a charged H-bond between E119 (loop) and R177 (lid) and a H-bond between N124(loop) and S180(lid). These interactions between the S-loop and lid are occasional in SEQ ID NO: 2 and SEQ ID NO: 3, rare in SEQ ID NO: 1 and SEQ ID NO: 7, and non-existent in SEQ ID NO: 4 and SEQ ID NO: 5.

The structures that have less interactions between the loop and lid have more opened loops and higher values of RMSF for the S-loop. Of these, SEQ ID NO: 5 has the lowest RMSFs for the lid.

Furthermore, for SEQ ID NO: 1 and SEQ ID NO: 7 an H-bond network is observed between the S-loop and the residues closer to the substrate binding pocket (Table 6). These have three consecutive H-bonds Y114:N34, N34:T299, T299:S50. For SEQ ID NO: 1, a T299:H38 hydrogen bond is also occasionally observed in one replica (22.5%). In SEQ ID NOs: 2, 5 and 6 and in two of the replicas of SEQ ID NOs: 3 and 4, the Y114:N34 H-bond is rare, and the network is compromised. In SEQ ID NO: 3, the T299:S50 H-bond is rare in two replicas.

In summary, the sequence differences in S-loop alter loop opening/closing. For SEQ ID NOs: 1, 3, 4, and 7, a network of H-bonds is observed from the S-loop to the substrate binding pocket. This network is not observed for SEQ ID NOs: 2, 5 and 6 in all the replicas.

TABLE 6
Hydrogen bond occurrence as a percentage of the simulation time. The averages and standard deviations for the three replicas
are reported. In the entries with standard deviations that are larger than the mean, values for the three replicas are reported. In
these cases, the replicas are sampling different conformations of the enzymes.
Hydrogen Bond Occurrence as a Percentage of the Simulation Time

| | K116:D131 | E119:R177 | N122:S180 | N124:S180 | S126:E182 | Y114:N34 | N34:T299 | T299:S50 |
|---|---|---|---|---|---|---|---|---|
| SEQ ID | 62.5% ± 9.1 | 0.0 | 1.0% ± 1.8 | 0.3/0.2/20.8% | — | 9.6% ± 5.8 | 38.2% ± 33.1 | 73.0% ± 7.6 |
| NO: 2 | | | | | | | | |
| SEQ ID | 57.1% ± 7.3 | 0.8% ± 1.3 | 0.8% ± 1.4 | 3.2% ± 1.9 | — | 42.4% ± 13.7 | 75.0% ± 33.0 | 56.8% ± 11.2 |
| NO: 1 | | | | | | | | |
| SEQ ID | 54.1% ± 25.2 | 1.7% ± 2.8 | 37.2%/0/0 | 12.3% ± 3.4 | — | 3.6/65.1/0.2% | 88.0% ± 10.2 | 6.1/17.3/86.1% |
| NO: 3 | | | | | | | | |
| SEQ ID | 52.1% ± 8.8 | 0.0 | 0.0 | 0.0 | — | 7.9/79.3/1.7% | 72.9% ± 34.9 | 85.4% ± 12.4 |
| NO: 4 | | | | | | | | |
| SEQ ID | 59.8% ± 2.0 | 0.0 | — | — | — | 1.9% ± 2.6 | 92.7% ± 4.6 | 79.4% ± 5.8 |
| NO: 5 | | | | | | | | |
| SEQ ID | 0.3% ± 0.5 | 68.2% ± 16.5 | 0.0 | 29.2% ± 17.5 | — | 1.8% ± 3.2 | 98.3% ± 1.8 | 75.4% ± 26.1 |
| NO: 6 | | | | | | | | |
| SEQ ID | 31.7% ± 5.9 | 4.4% ± 6.7 | 5.6% ± 7.0 | 1.3% ± 0.4 | 75.2/0/0.1 | 37.5% ± 13.7 | 66.7% ± 11.0 | 69.1% ± 7.4 |
| NO: 7 | | | | | | | | |

### Active Center—Molecular Docking

As mentioned above, the active center pocket of the studied VHPOs is 100% conserved. The homology models of VHPO enzymes were submitted to molecular docking (MD) simulations to better sample the conformations of the active center pockets and to more accurately describe differences between the enzymes. MD reference structures of all the enzymes, SEQ ID NOs: 1 to 7, were used for the molecular docking studies.

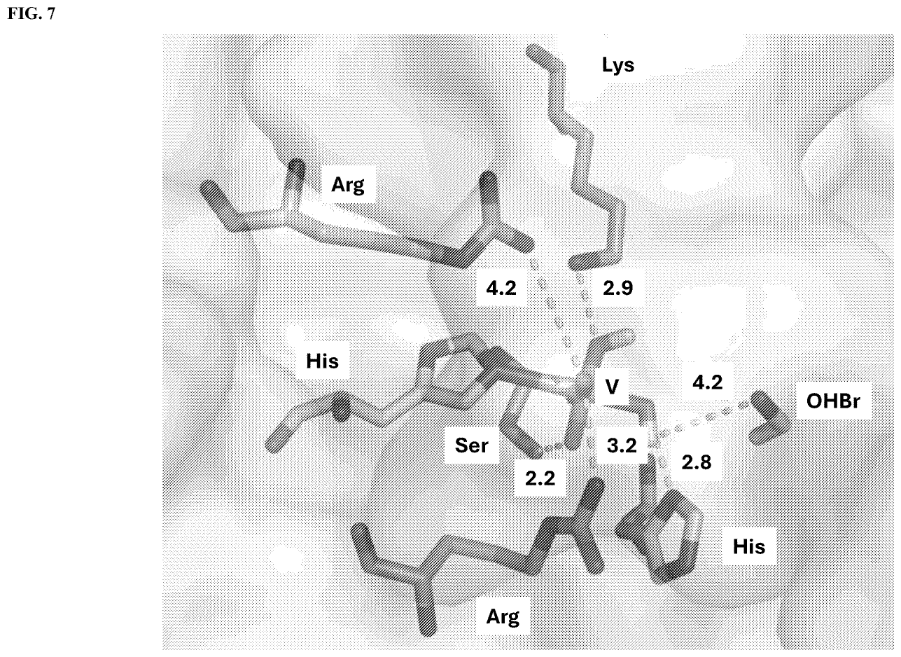

As shown in FIG. 7, the vanadium(V)-dioxo-dihydroxo is coordinated with a catalytic histidine and is stabilized by intermolecular interactions with a histidine, a lysine, a serine, and two arginine residues. OHBr was added to the model based on the interaction of Br with vanadate in crystal 7QWI (vanadium-dependent bromoperoxidase) found in the Worldwide Protein Data Bank. By comparing the size of the pocket of SEQ ID NO: 1 to that of SEQ ID NO: 2, it is possible to observe that SEQ ID NO: 2 has a considerably bigger pocket (FIG. 9). The disposition of a loop 287-297 (SEQ ID NO: 1 numbering) seems to be responsible for this difference.

It was decided to superimpose the structure of all enzymes to better characterize the size of the pockets (FIG. 8). SEQ ID NO: 1 and SEQ ID NO: 7 have a smaller pocket. SEQ ID NO: 3, SEQ ID NO: 4, and SEQ ID NO: 6 have the loop 287-297 in an intermediate position, while SEQ ID NO: 2 and SEQ ID NO: 5 have the loop in an open conformation which results in a larger pocket. These differences in the active pocket size have a substantial impact in the binding poses of the substrates.

Eleven substrates were docked to the VHPO enzymes of SEQ ID NOs: 1 to 7. These substrates were dimedone, acetylacetone, methyl pyruvate, oxaloacetic acid, dimethyl 1,3-acetonedicarboxylate, acetoacetic acid, methyl acetoacetate, malonic acid, malic acid, citric acid, and 3,3-dimethyl-1,5-cyclohexanediol. The dissociation constant (K_{d}) quantifies the binding affinity that a molecule has for an enzyme/receptor. Ligands that bind more tightly to the enzyme/receptor have a lower K_{d} value. Moreover, K_{d} corresponds to the concentration of substrate necessary so half of the protein molecules in the reaction are complexed with that substrate. On the other hand, the kinetic parameter K_{M} corresponds to the substrate concentration necessary to reach half of the maximum k_{cat}. When k_{cat}<<k₁ where k₋₁ represents the dissociation rate constant, the K_{M} and K_{d} are the same. For most cases k₋₁≤k_{cat}, so K_{M} is higher than K_{d}. Nevertheless, K_{d} and Kₘ are always correlated.

A higher binding affinity is an indication of a lower K_{M}. A shorter and/or a better angle of attack for a reaction coordinate of the rate limiting step is an indication of a higher k_{cat}. Table 7 summarizes all the docking results with the K_{d} and reaction coordinate distances highlighted. These two parameters together with the angle of attack and the percentage of the enol tautomeric form of the different substrates were taken into account when ordering the substrates in terms of catalytic efficiency.

The docking poses of dimedone, acetylacetone, methyl pyruvate and oxaloacetic acid were analyzed in the active center pockets of SEQ ID NO: 1, SEQ ID NO: 2 and SEQ ID NO: 5. SEQ ID NO: 1 corresponds to the most studied VHPO in the literature (Messerschmidt 1997). The Inventors had previously obtained high enzymatic activities with SEQ ID NO: 2. SEQ ID NO: 5 is the enzyme that most resembles SEQ ID NO: 2 in terms of overall stability and active center pocket conformation since the active center loop 286-297 is also placed in the open conformation contributing to a large active center pocket. The reaction coordinate associated with the nucleophilic attack of dimedone on OHBr is shorter for SEQ ID NO: 2 (4.6 Å) than SEQ ID NO: 5 (5.0 Å) and SEQ ID NO: 1 (5.1 Å). Dimedone binding affinity for all the three enzymes is high, being higher for SEQ ID NO: 5 (Kₐ=9.19 mM) than SEQ ID NO: 1 (K_{d}=20.23 mM) and SEQ ID NO: 2 (K_{d}=21.22 mM).

The reaction coordinate associated with acetylacetone nucleophilic attack on OHBr is shorter for SEQ ID NO: 1 (4.8 Å) and SEQ ID NO: 5 (4.9 Å) than SEQ ID NO: 2 (5.7 Å). Acetylacetone binding affinity is higher for SEQ ID NO: 5 (K_{d}=25.55 mM) than SEQ ID NO: 2 (K_{d}=54.56 mM) and SEQ ID NO: 1 (K_{d}=79.21 mM). The reaction coordinate associated with the methyl pyruvate nucleophilic attack of the substrate on OHBr is shorter for SEQ ID NO: 1 (4.2 Å) than SEQ ID NO: 5 (5.0 Å) and SEQ ID NO: 2 (6.4 Å). Methyl pyruvate binds strongly to the pocket. The binding affinity is higher for SEQ ID NO: 5 (K_{d}=23.12 mM) than SEQ ID NO: 2 (K_{d}=48.63 mM) and SEQ ID NO: 1 (K_{d}=84.69 mM). The reaction coordinate associated with the oxaloacetic acid nucleophilic attack of the substrate on OHBr is considerably shorter for SEQ ID NO: 5 (3.5 Å) than SEQ ID NO: 1 (5.1 Å) and SEQ ID NO: 2 (5.0 Å). Oxaloacetic acid binding affinity is higher for SEQ ID NO: 5 (K_{d}=29.57 mM) than SEQ ID NO: 2 (K_{d}=138.35 mM) and SEQ ID NO: 1 (K_{d}=169.58 mM).

The docking poses of substrates acetoacetic acid, methyl acetoacetate, dimethyl 1,3-acetonedicarboxylate and malonic acid were analyzed in the active center pockets of SEQ ID NO: 1, SEQ ID NO: 2 and SEQ ID NO: 5. The reaction coordinate associated with the acetoacetic acid nucleophilic attack of the substrate on OHBr is substantially shorter for SEQ ID NO: 5 (3.6 Å) than SEQ ID NO: 1 (5.2 Å) and SEQ ID NO: 2 (5.4 Å). The Acetoacetic acid binding affinity is higher for SEQ ID NO: 5 (K_{d}=36.01 mM) than SEQ ID NO: 2 (K_{d}=174.96 mM) and SEQ ID NO: 1 (K_{d}=171.23 mM). The reaction coordinate associated with the methyl acetoacetate nucleophilic attack of the substrate on OHBr is shorter for SEQ ID NO: 1 (4.4 Å) and SEQ ID NO: 5 (4.6 Å) than SEQ ID NO: 2 (5.5 Å). Methyl acetoacetate binding affinity is higher for SEQ ID NO: 5 (K_{d}=62.02 mM) than SEQ ID NO: 2 (K_{d}=183.09 mM) and SEQ ID NO: 1 (K_{d}=258.27 mM). The reaction coordinate associated with the dimethyl 1,3-acetonedicarboxylate nucleophilic attack of the substrate on OHBr is shorter for SEQ ID NO: 5 (4.1 Å) than SEQ ID NO: 1 (4.4 Å) and SEQ ID NO: 2 (5.6 Å). The reaction coordinate associated with the malonic acid nucleophilic attack of the substrate on OHBr is shorter for SEQ ID NO: 5 (3.5 Å) than SEQ ID NO: 1 (5.1 Å) and SEQ ID NO: 2 (5.9 Å). The malonic acid binding affinity is higher for SEQ ID NO: 5 (K_{d}=100.80 mM) than SEQ ID NO: 1 (K_{d}=270.52 mM) and SEQ ID NO: 2 (K_{d}=499.6 mM).

The docking poses of malic acid, citric acid and 3,3-dimethyl-1,5-cyclohexanediol were also analyzed. The reaction coordinate associated with the malic acid nucleophilic attack on OHBr is shorter for SEQ ID NO: 1 (3.9 Å) and SEQ ID NO: 5 (3.8 Å) than SEQ ID NO: 2 (5.2 Å). Malic acid binding affinity is higher for SEQ ID NO: 1 (K_{d}=238.56 mM) than SEQ ID NO: 2 (K_{d}=309.48 mM) and SEQ ID NO: 5 (K_{d}=350.93 mM). The reaction coordinate associated with the nucleophilic attack of the citric acid on OHBr is substantially shorter for SEQ ID NO: 5 (4.4 Å) and SEQ ID NO: 2 (4.6 Å) than SEQ ID NO: 1 (10.1 Å). The citric acid docking poses for SEQ ID NO: 1 and SEQ ID NO: 2 are not stable since binding affinity is 0. Citric acid can bind the pocket of SEQ ID NO: 5, and the pose seems to be catalytically relevant (K_{d}=236.25 mM). The 3,3-dimethyl-1,5-cyclohexanediol binding affinity for SEQ ID NO: 5 (K_{d}=6.47 mM), SEQ ID NO: 1 (K_{d}=7.49 mM) and SEQ ID NO: 2 (K_{d}=13.42 mM) is high. However, there is no C═C for the nucleophilic attack. It is possible that 3,3-dimethyl-1,5-cyclohexanediol can inhibit the enzyme.

### CONCLUSIONS

In this study, in silico tools were used to study the halogenation of dimedone, acetylacetone, oxaloacetic acid, dimethyl 1,3-acetonedicarboxylate, acetoacetic acid, methyl acetoacetate, malonic acid, malic acid, citric acid, 3,3-dimethyl-1,5-cyclohexanediol, and methyl pyruvate by seven VHPOs (i.e., SEQ ID NOs: 1 to 7). Initially, it was planned to run Molecular Dynamics of just the 4 most promising enzymes. However, since the active center of the seven VHPOs is 100% conserved, it was decided to run MD simulations of all the enzymes. MD analysis allowed accurate identification of the structural differences between the studied enzymes.

The largest variations on the overall structure of the enzymes are placed in the S-loop, in the active center pocket lid, and in the active center loop 286-297. The S-loop has the highest sequence variability between the enzymes. Enzymes with more interactions between the S-loop and the lid, like SEQ ID NOs: 2 and 6, have lower RMSFs (i.e., are more stable) for the loop and have more closed loops. On the other hand, enzymes with less interactions between the S-loop and the lid have a more open and flexible S-loop but might have a more stable lid. Accordingly, SEQ ID NO: 5, which has the shortest S-loop because of the deletion at position 117-120, has higher RMSFs for the S-loop than SEQ ID NO: 2 but has the most stable active center lid. SEQ ID NO: 2, SEQ ID NO: 6 and SEQ ID NO: 5 have the most stable loop/lid combinations of all enzymes.

Loop and lid opening and closing could influence enzyme stability. Furthermore, the S-loop conformational change seems to be related to the substrate binding pocket. Accordingly, a network of H-bonds involving residues Y114, N34, T299, S50 is observed for SEQ ID NOs: 1, 3 4 and 7, but not for SEQ ID NOs: 2, 5 and 6 in all the replicas.

The SEQ ID NO: 1 and SEQ ID NO: 7 VHPOs have a different active center loop 286-297 disposition which leads to a small active center pocket. SEQ ID NO: 3, SEQ ID NO: 4 and SEQ ID NO: 6 have the active center loop 286-297 in an intermediate position, while SEQ ID NO: 2 and SEQ ID NO: 5 have the active center loop in an open conformation which results in a larger active center pocket. The eleven substrates were docked to the reference structures of the MDs to obtain substrate:enzyme Michaelis-Menten complexes. The larger active center pockets of SEQ ID NO: 2 and SEQ ID NO: 5 can better accommodate the analyzed substrates.

The substrates were ranked according to: 1) binding affinity; 2) binding pose; and 3) substrate reactivity (Table 7). For SEQ ID NO: 2, the rank is the following: dimedone>acetylacetone>methyl pyruvate>oxaloacetic acid>acetoacetic acid>dimethyl 1,3-acetonedicarboxylate>methyl acetoacetate>malonic acid>malic acid>citric acid>3,3-dimethyl-1,5-cyclohexanediol. For SEQ ID NO: 5, the rank is the following: dimedone>methyl pyruvate>acetoacetic>acid acetylacetone>oxaloacetic acid>dimethyl 1,3-acetonedicarboxylate>malonic acid>methyl acetoacetate>citric acid>malic acid>3,3-dimethyl-1,5-cyclohexanediol. It is possible that 3,3-dimethyl-1,5-cyclohexanediol behaves like competitive inhibitor of the other substrates. Citric acid cannot bind the active center pocket of SEQ ID NO: 1, SEQ ID NO: 2, SEQ3 and SEQ ID NO: 7. However, this analysis indicates that citric acid binds the pocket of SEQ ID NO: 5 with a good affinity and in a catalytically relevant pose.

TABLE 7
Docking results for SEQ ID NOs. 1-7.

| Substrates Listed in | SEQ1: Substrate | SEQ1: Substrate | Substrates Listed in | SEQ2: Substrate | SEQ2: Substrate |
|---|---|---|---|---|---|
| Descending Order Based | Dissociation | Reaction | Descending Order Based | Dissociation | Reaction |
| on Catalytic Efficiency | Constant (mM) | Coordinate (Å) | on Catalytic Efficiency | Constant (mM) | Coordinate (Å) |
| dimedone | 20.23 | 5.1 | dimedone | 21.22 | 4.6 |
| acetylacetone | 79.21 | 4.8 | acetylacetone | 54.56 | 5.7 |
| methyl pyruvate | 84.69 | 4.2 | methyl pyruvate | 48.63 | 6.4 |
| oxaloacetic acid | 169.58 | 5.1 | oxaloacetic acid | 138.35 | 5.0 |
| dimethyl 1,3- | 573.79 | 4.4 | acetoacetic acid | 171.23 | 5.4 |
| acetonedicarboxylate | | | | | |
| acetoacetic acid | 174.96 | 5.2 | dimethyl 1,3- | 490.06 | 5.6 |
| | | | acetonedicarboxylate | | |
| methyl acetoacetate | 258.27 | 4.4 | methyl acetoacetate | 183.09 | 5.5 |
| malonic acid | 270.52 | 5.1 | malonic acid | 499.96 | 5.9 |
| malic acid | 238.56 | 3.9 | malic acid | 309.48 | 5.2 |
| citric acid | 0 | 10.1 | citric acid | 0 | 4.6 |
| 3,3-dimethyl-1,5- | 7.49 | 5.2 | 3,3-dimethyl-1,5- | 13.42 | 4.6 |
| cyclohexanediol | | | cyclohexanediol | | |
| Substrates Listed in | SEQ3: Substrate | SEQ3: Substrate | Substrates Listed in | SEQ4: Substrate | SEQ4: Substrate |
| Descending Order Based | Dissociation | Reaction | Descending Order Based | Dissociation | Reaction |
| on Catalytic Efficiency | Constant (mM) | Coordinate (Å) | on Catalytic Efficiency | Constant (mM) | Coordinate (Å) |
| dimedone | 20.22 | 6.9 | dimedone | 7.45 | 8.2 |
| methyl pyruvate | 78.98 | 5.9 | acetylacetone | 13.79 | 7.3 |
| acetylacetone | 54.60 | 6.3 | methyl pyruvate | 13.20 | 7.4 |
| acetoacetic acid | 397.92 | 5.9 | acetoacetic acid | 71.89 | 6.9 |
| dimethyl 1,3- | 400.60 | 4.7 | dimethyl 1,3- | 144.81 | 6.7 |
| acetonedicarboxylate | | | acetonedicarboxylate | | |
| oxaloacetic acid | 626.05 | 4.8 | oxaloacetic acid | 110.09 | 8.3 |
| methyl acetoacetate | 165.72 | 6.6 | methyl acetoacetate | 47.95 | 7.1 |
| malic acid | 280.80 | 8.3 | malic acid | 389.01 | 5.3 |
| malonic acid | 438.87 | 5.8 | malonic acid | 370.16 | 7.3 |
| citric acid | 0 | 4.8 | citric acid | 775.15 | 4.2 |
| 3,3-dimethyl-1,5- | 26.39 | 5.7 | 3,3-dimethyl-1,5- | 3.00 | 8.3 |
| cyclohexanediol | | | cyclohexanediol | | |
| Substrates Listed in | SEQ5: Substrate | SEQ5: Substrate | Descending Order Based | SEQ6: Substrate | SEQ6: Substrate |
| Descending Order Based on | Dissociation | Reaction | Substrates Listed in | Dissociation | Reaction |
| Catalytic Efficiency | Constant (mM) | Coordinate (Å) | on Catalytic Efficiency | Constant (mM) | Coordinate (Å) |
| Dimedone | 9.19 | 5.0 | Dimedone | 22.52 | 6.0 |
| Methyl pyruvate | 23.12 | 5.0 | Acetylacetone | 61.49 | 5.6 |
| Acetoacetic acid | 36.01 | 3.6 | Methyl pyruvate | 62.95 | 6.6 |
| Acetylacetone | 25.55 | 4.9 | Acetoacetic acid | 160.88 | 5.6 |
| Oxaloacetic acid | 29.57 | 3.5 | Oxaloacetic acid | 244.18 | 4.2 |
| Dimethyl 1,3- | 68.66 | 4.1 | Dimethyl 1,3- | 246.03 | 5.6 |
| acetonedicarboxylate | | | acetonedicarboxylate | | |
| Malonic acid | 100.80 | 3.5 | Methyl acetoacetate | 169.41 | 5.7 |
| Methyl acetoacetate | 62.02 | 4.6 | Malonic acid | 381.71 | 5.7 |
| Citric acid | 236.25 | 4.4 | Malic acid | 233.60 | 6.7 |
| Malic acid | 350.93 | 4.1 | Citric acid | 835.08 | 6.1 |
| 3,3-dimethyl-1,5- | 6.47 | 3.6 | 3,3-dimethyl-1,5- | 20.14 | 3.5 |
| cyclohexanediol | | | cyclohexanediol | | |

| Substrates Listed in Descending | SEQ7: Substrate | SEQ7: Substrate |
|---|---|---|
| Order Based on Catalytic | Dissociation | Reaction |
| Efficiency | Constant (mM) | Coordinate (Å) |
| Acetylacetone | 57.77 | 3.4 |
| Methyl pyruvate | 57.86 | 3.9 |
| Dimedone | 28.62 | 6.3 |
| Acetoacetic acid | 224.51 | 3.8 |
| Dimethyl 1,3-acetonedicarboxylate | 470.36 | 3.8 |
| Methyl acetoacetate | 226.80 | 3.8 |
| Oxaloacetic acid | 214.48 | 5.7 |
| Malonic acid | 233.23 | 8.3 |
| Malic acid | 455.41 | 7.2 |
| Citric acid | 0 | 5.6 |
| 3,3-dimethyl-1,5-cyclohexanediol | 18.13 | 6.1 |

### Example 5. Expression and Purification of VHPO Proteins in Pichia pastoris

### Background

A review of the relevant literature indicates that the VHPO enzymes are generally expressed in fungi as intracellular proteins. However, substantial VHPO activity can be detected outside of the hyphae in the growth media of the fungal cultures (Hemrika et al., 1999). Nevertheless, neither apparent N-terminal leader peptides ensuring secretory properties of eukaryotic proteins have been described in any fungal VHPOs nor have such leaders been identified by Signal IP (Teufel et al., 2022) during analysis of the target VHPOs (i.e., SEQ ID NOs: 2-7). The pathway and mechanism of the fungal secretion of the VHPO enzyme remains unclear. Hemrika et al. (1999) suggested that fungi possess some unconventional secretory pathway.

There are numerous publications describing heterologous expression of various peroxidases in yeast. Both, classical Saccharomyces cerevisiae and so called non-conventional yeast Pichia pastoris have been proven as good hosts for expression of the peroxidases, mostly as secreted soluble proteins (Wang et al., 2004, Ryu et al., 2008, Kim et al, 2009, Krainer et al., 2016, Wen et al., 2017, Bronikowski et al., 2018). Undoubtedly, the secretory expression is considered as a preferable approach for production of a protein through bioprocessing since it allows for much simpler purification technologies without the necessity of cell lysis and separation of the target proteins from cell debris and multiple cellular proteins.

One publication describes a successful intracellular expression in the yeast S. cerevisiae of a Curvularia inaequalis chloroperoxidase 1VNC (Hemrika et al., 1999). The recombinant yeast strain was able to express the 1VNC chloroperoxidase as a cytosolic protein which was easily purified from the lysed cells with a relatively high yield of 100 mg from 1 L culture. One important observation from the publication is that the expressed intracellularly chloroperoxidase can be purified as an enzymatically inactive apoenzyme, which can then easily be activated in vitro by supplying the vanadium ions (orthovanadate) to the protein solution. Another important observation is a relatively high stability of the expressed protein and its resistance to proteolysis. Interestingly, the extracted yeast protein tended to form high molecular weight multi-molecular aggregates. The authors attempted to construct a yeast strain secreting the chloroperoxidase by fusion of the protein with a secretion leader, yeast pre-pro alpha mating factor peptide. However, this established approach for secretory expression of heterologous proteins in yeast by introduction to the N-terminus of a protein a leader peptide was unsuccessful in case of the 1VNC chloroperoxidase.

It is important to underscore that there are high degrees of identity (~95%) and similarity (>95%) between the Curvularia inaequalis 1VNC chloroperoxidase and each of the VHPO enzymes chosen for heterologous expression in yeast. The amino acid sequences of the chosen VHPOs were analyzed by multiple protein alignment to evaluate the amino acid variability among the proteins (FIG. 1). Since the alignment clearly shows high similarity of the proteins to each other and to the 1VNC chloroperoxidase, it was decided to construct P. pastoris recombinant strains expressing these VHPO proteins intracellularly followed by extraction of soluble proteins from the fungal biomass of the recombinant strains.

### Materials and Methods

Construction of Pichia pastoris Recombinant Cell Lines

Purification of an intracellularly expressed protein requires an efficient method of separation of a target protein from other cellular proteins. A commonly established approach to simplify purification of a soluble protein and to increase the yield of the product is the fusion of a protein with an affinity tag. One such tag is the His-tag, being attached to either terminus of a protein enabling an efficient purification with special resins. According to available information regarding the structures of VHPO as well as the computational models of folding of the chosen VHPOs, neither terminal region of the protein possesses any apparent function nor influences the protein folding or formation of the enzyme's active site. Based on these assumptions, it was decided to attach a 6×His tag to the N-termini of the proteins. In addition to that, the TEV-protease tag was introduced between the 6×His-tag and the second amino acid residue of the molecules. Such a structural modification of the expressed VHPOs wouldn't alter the properties and enzymatic activities of the expressed enzymes but would enable an affinity purification of the proteins. The presence of the TEV-tag allows the total removal of both tags by treatment of the protein preparations with TEV-protease when it is needed.

There are two general fermentation technologies for expression of heterologous proteins in the yeast P. pastoris. One is based on the expression controlled by a strong regulated promoter of the P. pastoris gene AOX1. The second is unregulated expression controlled by various strong promoters of the P. pastoris genes which are transcribed constitutively in the host cells. In most cases, the level of protein expression under control of the AOX1 promoter is higher than that with a constitutive promoter. However, there are certain disadvantages of induction by methanol expression, particularly a longer fermentation time and the complexity of the process requiring switching of the regimens with replacement of carbon sources when the final induction is performed with methanol, a highly toxic and flammable compound. From this point of view, a simpler and shorter unregulated process might be considered as the technology of choice in cases where the expressed protein does not influence the fitness of the culture and decent levels of protein expression under control of a constitutive promoter can be achieved.

To make a choice of which expression technology is more suitable for manufacturing of the VHPOs, during the initial stage of the project's execution two expression vectors with methanol regulated promoter AOX1 and the constitutive promoter UPP were used for expression of each VHPO gene. In order to reduce the methanol and oxygen consumptions during the induction phase for the P. pastoris recombinant strains expressing the target genes under control of the AOX1 promoter, an aox1-delta1 P. pastoris strain was used as a recipient for expression of the VHPO proteins.

For intracellular expression of the proteins, the optimized DNA sequences encoding the chosen proteins with attached N-terminal 6×His and TEV tags were synthesized and cloned into two expression vectors, pJAZ and pJUZ. Both vectors are expression vectors for intracellular expression with a zeocin selectable marker for selection of yeast colonies with recombinant plasmids integrated into the genome of the recipient strain. The pJAZ vector contains a methanol inducible promoter AOX1 and does not express a target protein during the growth phase but allows the expression only under methanol induction. The pJUZ vector contains a constitutive promoter UPP which drives the transcription of a downstream open reading frame (i.e., gene) during proliferation of the culture in the presence of any carbon source.

The recipient Pichia pastoris strains were transformed with the constructed recombinant plasmids. Numerous clones expressing VHPO proteins were selected. An enzymatic assay was developed to demonstrate the VHPO enzymatic activities in the cells of constructed strains. Six P. pastoris strains (cells lines) constitutively expressing each target protein (enzyme) were selected for large-scale protein purification.

Purification of VHPO Enzymes from P. pastoris Cell Lines

The cells of selected Pichia pastoris strains expressing the target VHPO enzymes were grown as large-scale flask cultures. The expression of enzyme was demonstrated before the cells were used for purification of the VHPO proteins. For purification of the enzymes from the cells, the biomass of cells was resuspended in equal mass of HisTrap A buffer (2×PBS pH 7.4+250 mM NaCl) containing HALT protease inhibitor mix (Thermo Fisher) without EDTA, and the cells were broken by glass beads using a BioSpec Beadbeater, six 1 min bead beating cycles with 5+ minute rest time between breakage runs.

Beads were washed extensively with HisTrap A+HALT to remove all cell extract, which was pooled as the final cell extract. The total cell extracts were spun at 30,000×g for 30 minutes at 4° C. Semi-clarified supernatants were filtered through a 500 kDa cutoff filter on a SPECTRUM KROSFLO® system. The retained debris was washed with an additional volume of HisTrap A buffer.

The combined filtrates were concentrated using cutoff filters, either directly by a 3 kDa filters or sequentially by 30 kDa and then 3 kDa filter. Concentrated material (~80 ml) was loaded onto a 10 ml HisTrap column (Cytiva) at 1 ml/min with mixing in of 2% HisTrap B buffer (HisTrap A+500 mM imidazole) prior to column input. The column was washed to a UV 280 baseline with 5 column volumes of 98% HisTrap A, 2% HisTrap B at 2 ml/min. Gradient elution was carried out over 20 column volumes from 0%-100% HisTrap B at 2 ml/min. ¼ column volume fractions were collected, and appropriate fractions were analyzed by SDS-PAGE. Pooled fractions were dialyzed over 36 hours at 6° C. against 4 changes (>20 volumes) of 50 mM HEPES buffer pH 7.4.

Final volumes of collected protein solutions were mixed with 75% glycerol to make a final glycerol concentration of 15% (4 volumes protein solution to 1 volume 75% glycerol), and vanadate (NEB) was added to the final concentration of 1 mM. The protein solutions were aliquoted, frozen, and stored at −80° C.

### Example 6. Kinetic Study of the Purified Vanadium-Dependent Haloperoxidases

### Materials and Methods

Kinetic parameters of VHPOs were determined using the monochlorodimedone (MCD) (2-chloro-5,5-dimethyl-1,3-dimedon) assay. The assay was carried out in a 200 μL reaction volume using Greiner UV-Star® 96 well plates. MCD conversion was spectrophotometrically monitored at 290 nm (F=20/mM·cm) using a CLARIOstar® Plus microplate reader (BMG labtech, Germany) at 25° C. The optimal pH of enzyme activity for each VHPO was characterized under a typical MCD assay (KBr 5 mM, Na₃VO₄ 0.1 mM, MCD 0.05 mM, H₂O₂ 4.6 mM, and a corresponding buffer 50 mM) for VHPO. The buffers were acetate buffer for pH 4-5.5, MES buffer for pH 6 and 6.5, and HEPES buffer for pH 7-8. The MCD mixture was prepared and aliquoted to 96-well plates, followed by enzyme addition (10 μL of appropriate dilution). The reaction was initiated by injecting H₂O₂. Following the optimal pH condition, kinetic parameters for KBr and H₂O₂ were determined. Stock solutions were prepared in advance for KBr (0.25M), Na₃VO₄ (25 mM), MCD (0.5 mM, adjusted pH to 6), and buffers (1M).

The absorbance values were plotted against time. For each curve, the slope of linear regression was determined as the initial velocity (Abs/min) of the enzyme activity. These initial velocity values were fitted against the concentrations of KBr or H₂O₂ using the enzyme kinetic category with substrate inhibition model (OriginPro 2024) to obtain the maximum velocity (Vₘₐₓ), Michaelis-Menten constant (K_{M}) and inhibition constant (K₁).

The following enzyme concentrations were used in the MCD assay:
For Curvularia sp. 4388 (NMI Accession No. V22/011149) liquid cultures, 10 μL of supernatant (sterile filtered by 0.2 μm filter paper) was added into a 200 μL MCD assay making a final 20-fold dilution of supernatant.
For purified VHPO from a P. pastoris recombinant cell line expressing SEQ ID NO: 2, 10 μL of a 10-fold dilution in pure water of the purified VHPO was used in 200 μL MCD assay resulting in a final 200-fold dilution.
For purified VHPOs from P. pastoris recombinant cell lines expressing SEQ ID NO: 3, 5, or 7, 20 μL of a 2-fold dilution in pure water of the purified VHPO was used in 200 μL MCD assay resulting in a final 20-fold dilution.

### Results

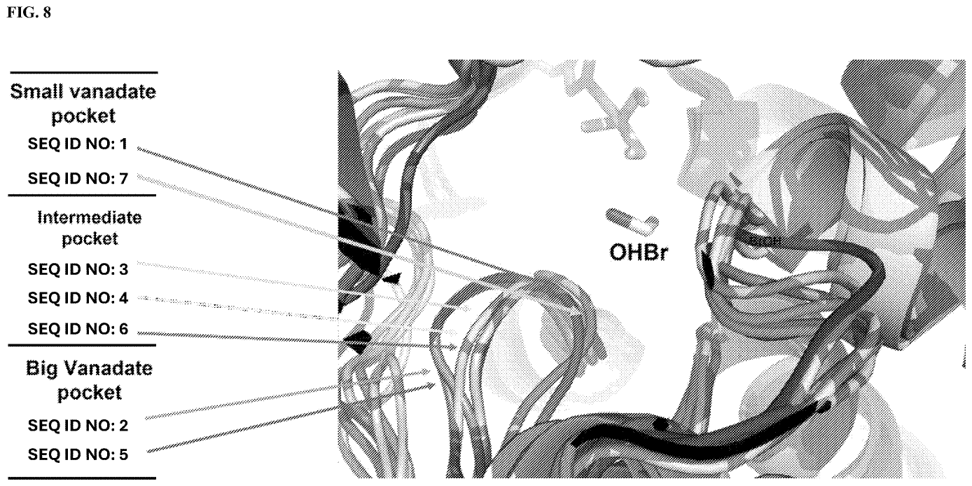

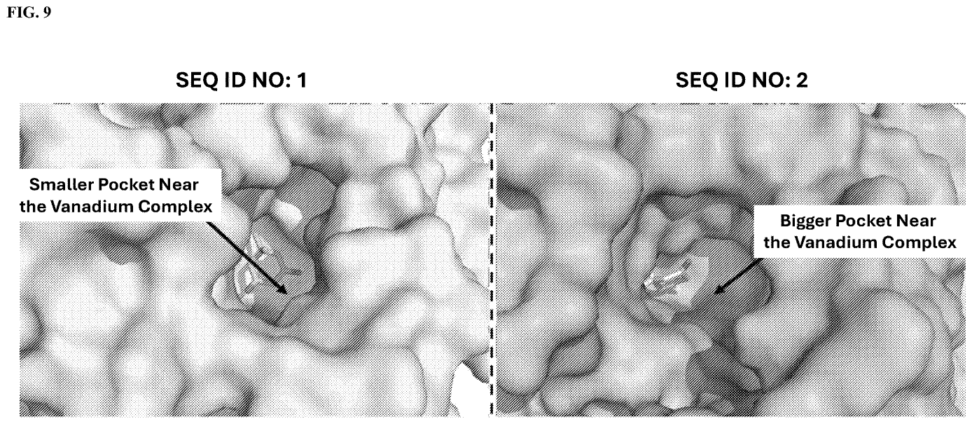

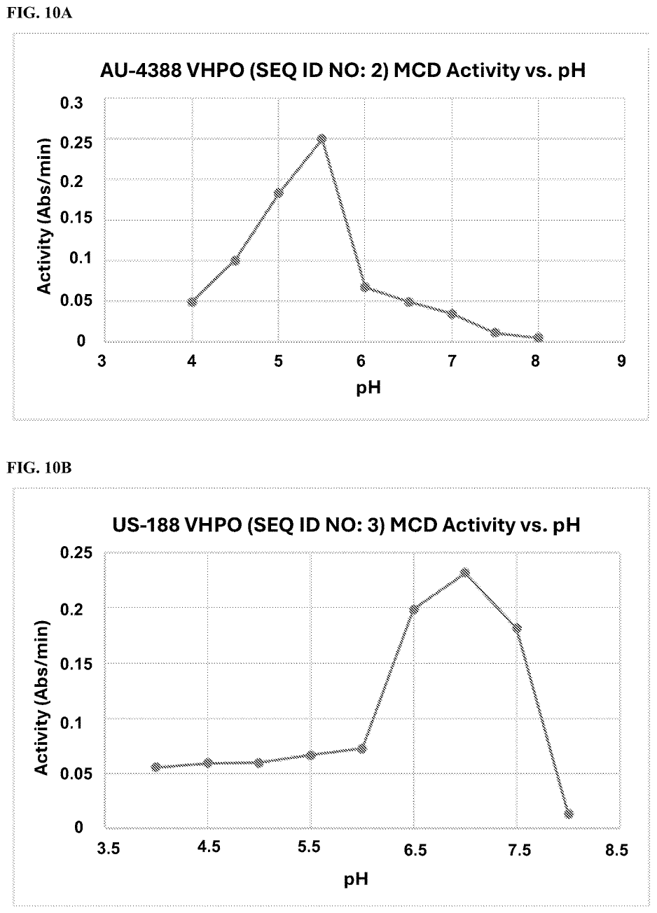

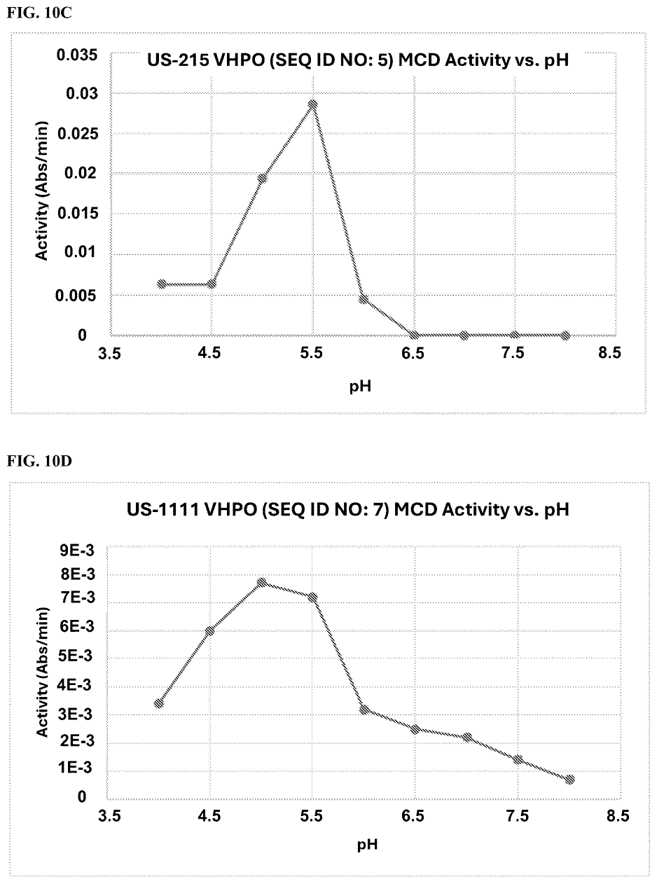

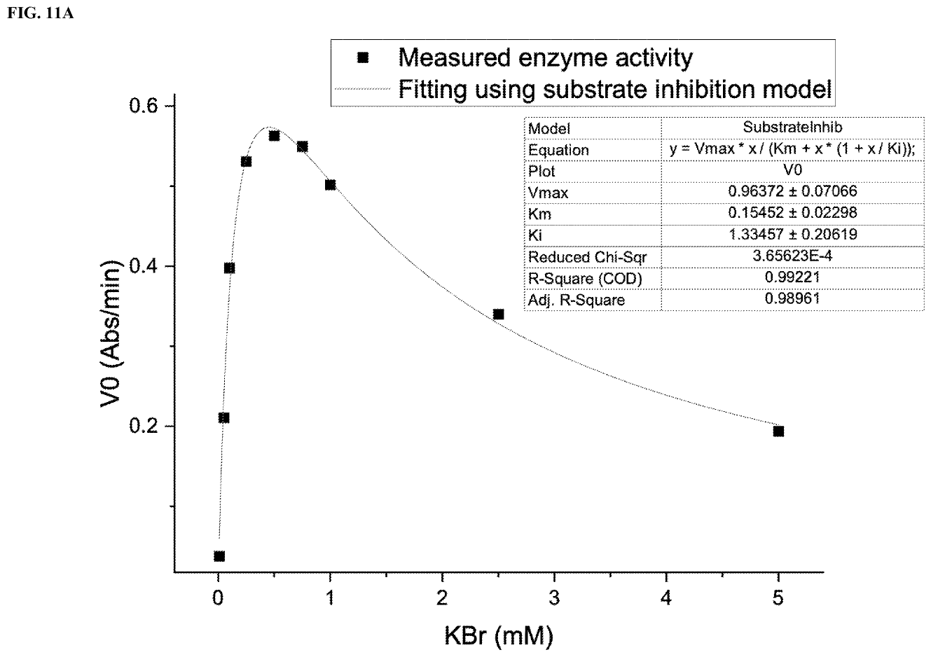

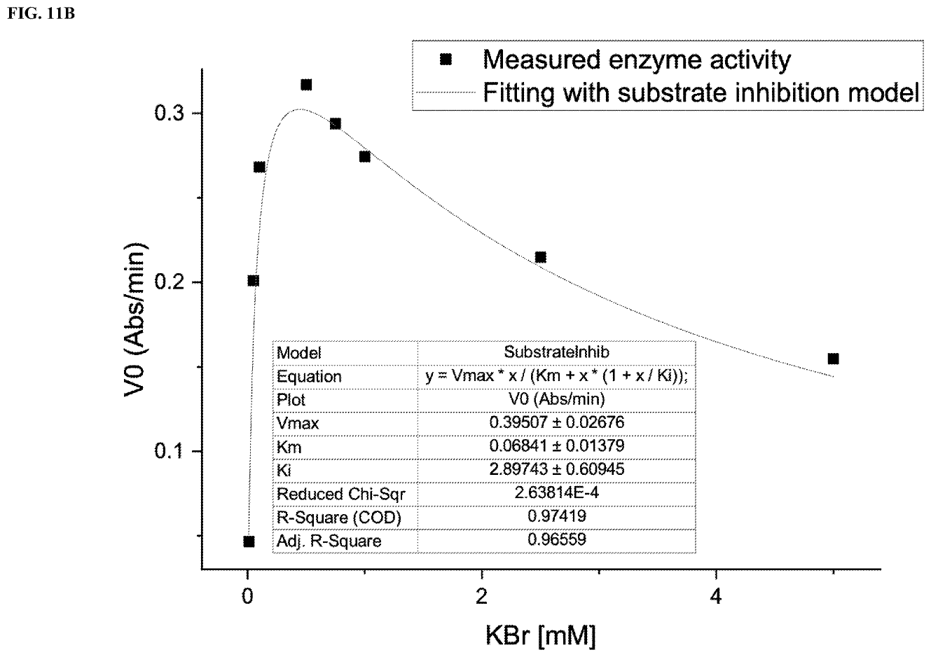

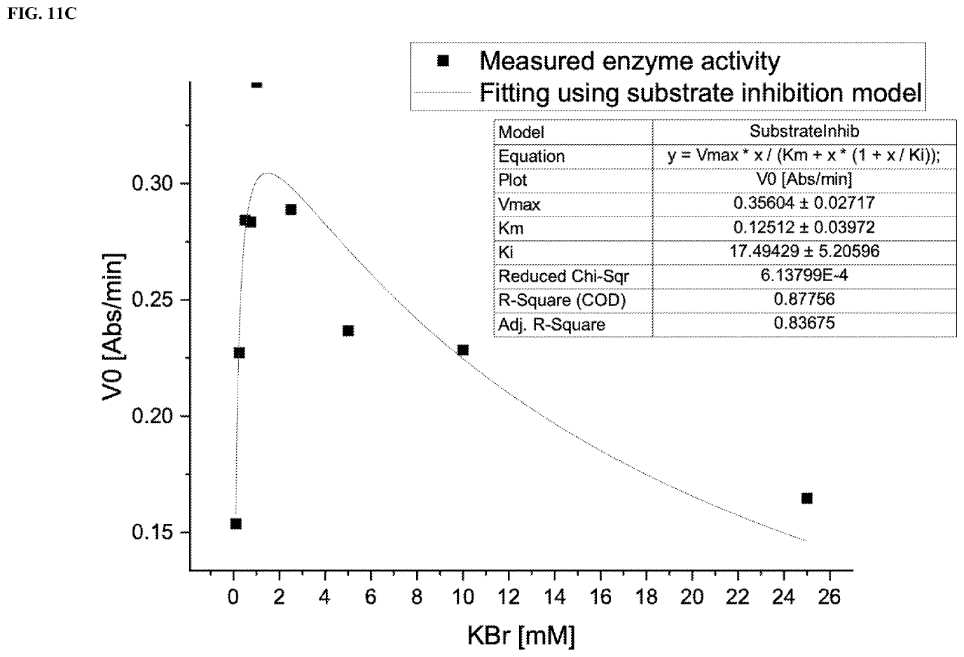

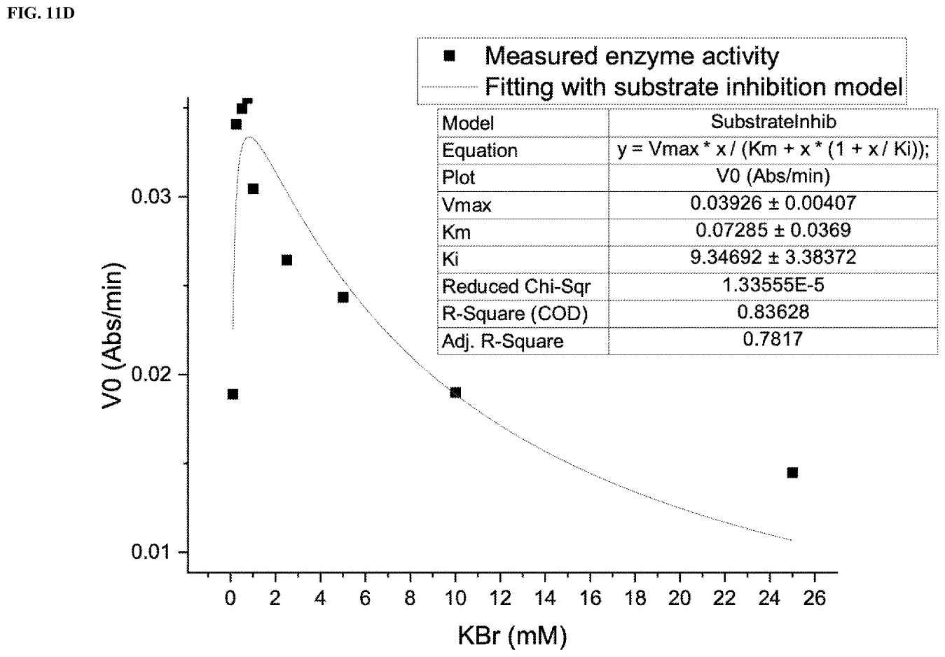

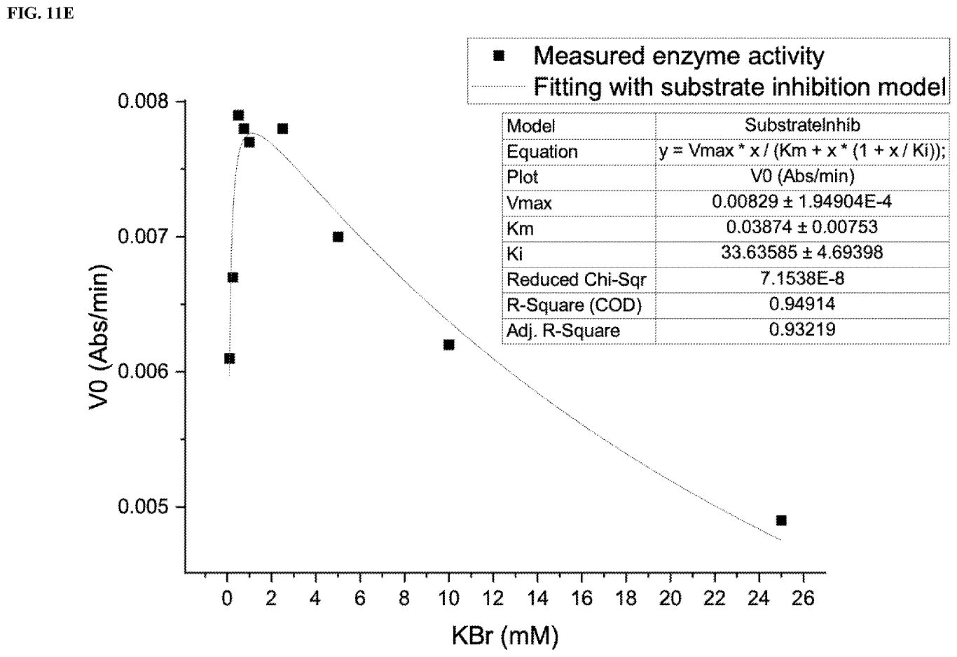

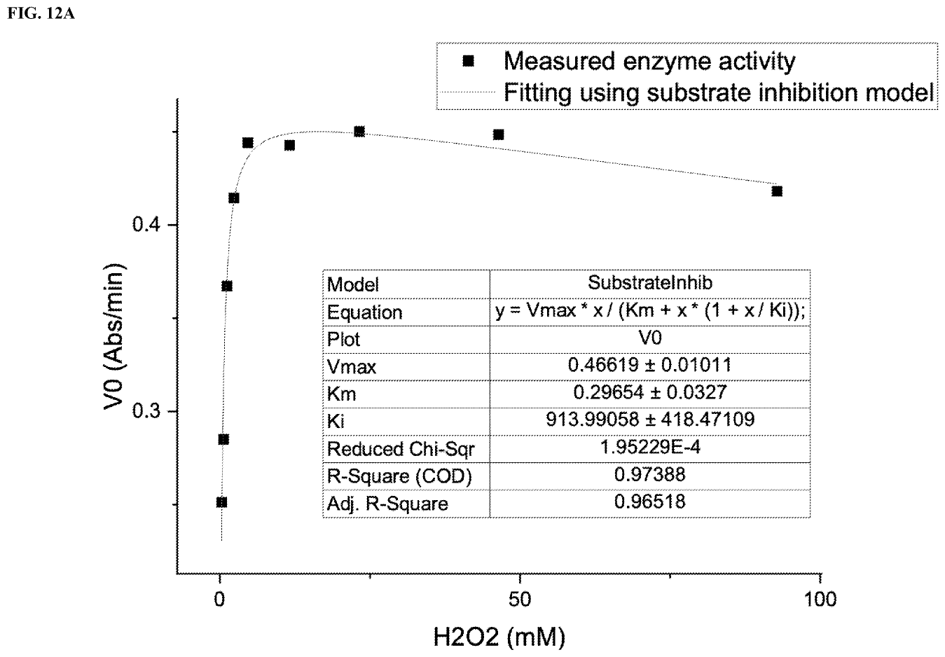

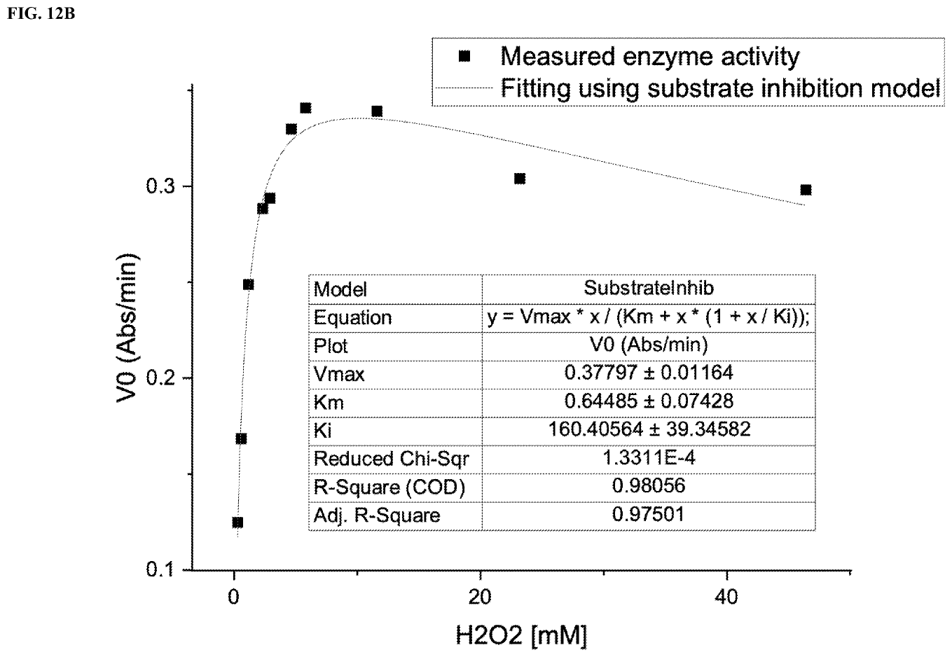

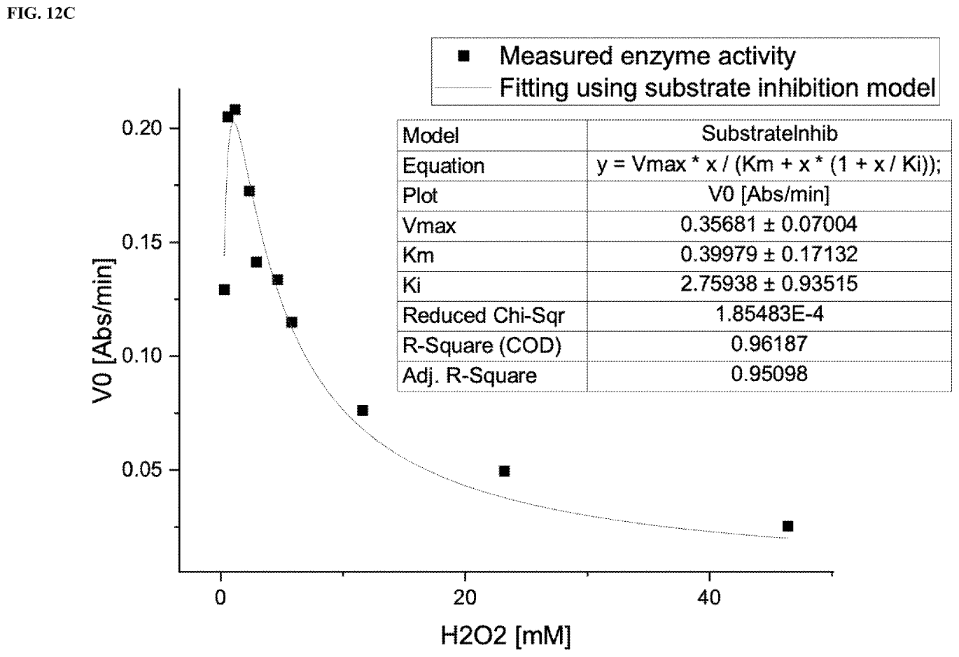

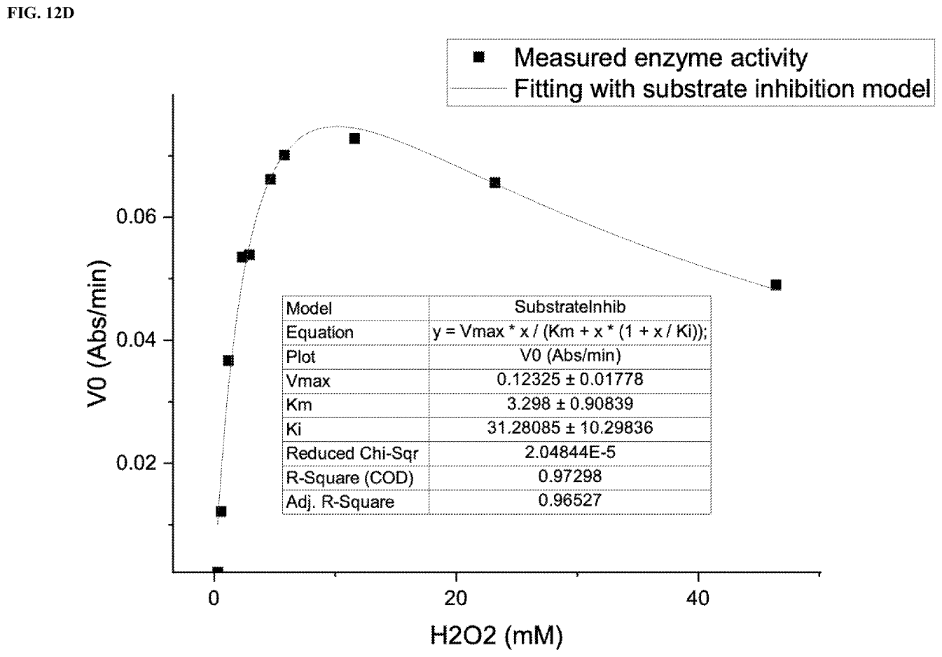

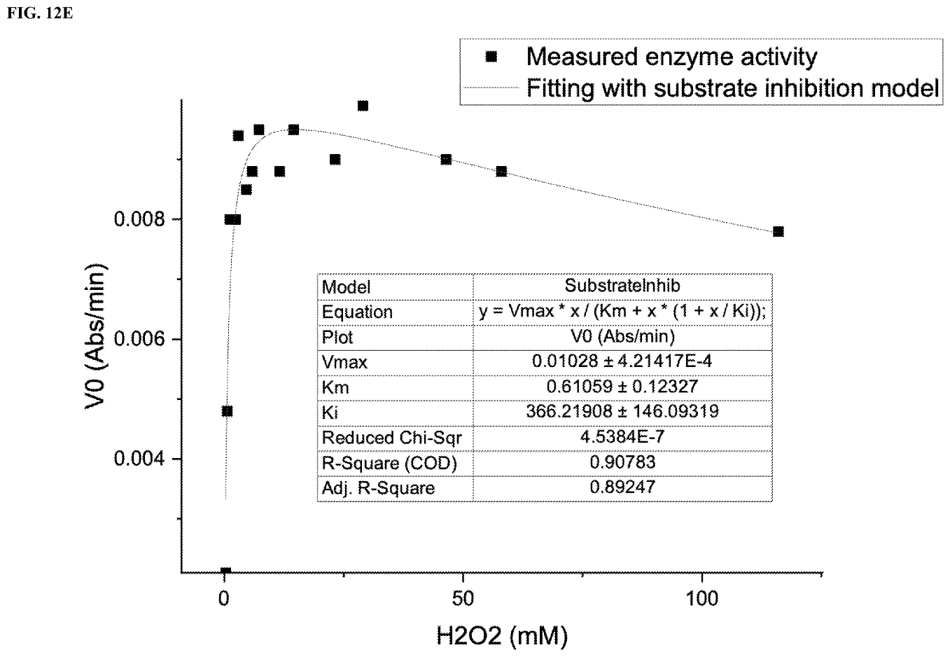

The optimal pH for the VHPOs expressed in Curvularia sp. 4388 (NMI Accession No. V22/011149) (i.e., SEQ ID NO: 2), Curvularia spicifera US-188 (ATCC Accession No. PTA-127510) (i.e., SEQ ID NO: 3), Curvularia pseudobrachyspora US-215 (ATCC Accession No. PTA-127509) (i.e., SEQ ID NO: 5), and Curvularia protuberata US-1111 (ATCC Accession No. PTA-127607) (i.e., SEQ ID NO: 7) were determined to be 5.5, 7, 5.5, and 5, respectively (see FIGS. 10A, 10B, 10C, and 10D). The VHPO kinetic activity against KBr concentrations and against H₂O₂ concentrations were experimentally determined with Curvularia sp. 4388 (NMI Accession No. V22/011149) culture supernatant at pH 5.5, purified VHPO from Curvularia sp. 4388 (NMI Accession No. V22/011149) (i.e., SEQ ID NO: 2) at pH 5.5, purified VHPO from Curvularia spicifera US-188 (ATCC Accession No. PTA-127510) (i.e., SEQ ID NO: 3) at pH 6.5, purified VHPO from Curvularia pseudobrachyspora US-215 (ATCC Accession No. PTA-127509) (i.e., SEQ ID NO: 5) at pH 5.5, and purified VHPO from Curvularia protuberata US-1111 (ATCC Accession No. PTA-127607) (i.e., SEQ ID NO: 7) at pH 5.5 (see FIGS. 11A-11E, FIGS. 12A-12E, and Table 8).

TABLE 8
Kinetic parameters of VHPOs from Curvularia spp. determined with the MCD assay at optimal pH of enzyme
activity for each protein.

| | AU-4388 | Purified VHPO | Purified VHPO | Purified VHPO | Purified VHPO |
|---|---|---|---|---|---|
| | Supernatant | (SEQ ID NO: 2) | (SEQ ID NO: 3) | (SEQ ID NO: 5) | (SEQ ID NO: 7) |
| Optimal pH | pH 5.5 | pH 5.5 | pH 6.5 (pH 7) * | pH 5.5 | pH 5.5 (pH 5) ** |
| K_{M} (Br-) | 0.155[mM] | 0.068 [mM] | 0.125 [mM] | 0.073 [mM] | 0.0387 [mM] |
| K_{I} (Br-) | 1.335 [mM] | 2.897 [mM] | 17.494 [mM] | 9.347 [mM] | 33.636 |
| Vₘₐₓ (Br-) | 0.964 [Abs/min] | 0.395 [Abs/min] | 0.356[Abs/min] | 0.039 [Abs/min] | 0.0083 [Abs/min] |
| K_{M} (H₂O₂) | 0.297 [mM] | 0.645 [mM] | 0.40 [mM] | 3.298 [mM] | 0.611 [mM] |
| K_{I} (H₂O₂) | 913.99 [mM] | 160.41 [mM] | 2.759 [mM] | 31.281 [mM] | 366.219 [mM] |
| Vₘₐₓ (H₂O₂) | 0.466 [Abs/min] | 0.378 [Abs/min] | 0.357 [Abs/min] | 0.123 [Abs/min] | 0.0103 [Abs/min] |

* Although US-188 VHPO (SEQ ID NO: 3) demonstrates an optimal activity at pH 7, the enzyme activity was inhibited by H₂O₂ at this pH, particularly at increasing H₂O₂ concentrations (Soedjak et al., 1995). A compatible high activity of US-188 VHPO can be achieved at pH 6.5 (FIG. 10B), so kinetic parameters of US-188 were determined at this pH 6.5.
** The optimal pH for US-1111 VHPO (SEQ ID NO: 7) is between 5 and 5.5 (FIG. 10D). Kinetic analysis was conducted at pH 5.5 to make it more comparable with other sequences (i.e., AU-4388 VHPO (SEQ ID NO: 2) and US-215 VHPO (SEQ ID NO: 5).

### Example 7. Testing Alternative Substrates for their Utility as Bromoform Precursors Using a VHPO Catalyzed Pathway

### Background

The purpose of this experiment is to investigate the potential use of various compounds such as citric acid, sucrose and methyl pyruvate to produce bromoform using a reaction catalyzed by a fungal VHPO. Previous research has indicated that the reaction between citric acid and hypochlorous acid (HOCl) to form chloroform could potentially be extended to hypobromous acid (HOBr), resulting in the synthesis of bromoform from citric acid. Blatchley et al. 2003 provides further information regarding the use of Cu²⁺ to catalyze this reaction. Other alternative substrates, which share structural similarities to various diketone and methyl ketones precursors, were also analyzed. Substrates where alcohol groups could be oxidized to form ketones in situ were considered as well. The aim was to evaluate these alternatives with both a chemical route using aqueous bromine solution and later a VHPO enzyme catalyzed route to form bromoform (CHBr₃). A limitation of this work is that gas chromatography-mass spectrometry (GCMS) is only able to identify the final product (i.e., bromoform) and cannot be used to quantify or characterize the intermediates. In situ solution state NMR experiments could be used to overcome these shortcomings.

### Materials and Methods

### Buffers

For developing and optimizing the reaction pathway, different buffer systems (at varying pH values) were assessed to find an optimal one. The diverse substrates that were believed to have potential were prepared in a 10 mM solution in different buffer systems. Additionally, dimedone was also used as a positive control and tested using three different buffer systems for both the chemical and enzyme routes. The three buffer systems evaluated were: 0.2M HEPES, pH 7; 0.2M potassium phosphate buffer, pH 7; and 0.2M sodium citrate monobasic, pH 7. The substrate evaluated in the reactions were: dimedone (as positive control), citric acid, sucrose, malic acid, ethyl acetoacetate, levulinic acid, ethyl lactate, lactic acid, 3-hydroxybutyric acid, 1,3-cyclohexanediol, acetone-dicarboxylic acid, malonic acid, pyruvic acid, and methyl pyruvate. 10 mM solutions of all substrates were prepared.

Ultimately, HEPES pH 7 was used as the buffer. This was done as PBS was seen to moderately inhibit the reaction when performed with the positive control dimedone. Citric acid has been used as a buffer system for monochlorodimedone assays; however, it was decided not to use it as a buffer here as trace level conversion of bromoform were observed when using it without any substrate.

### Chemical Reaction (Hypobromous Acid, HOBr/Bromine Water Addition)

For the chemical reaction of bromine water with the 10 mM substrate solutions, 20 mL headspace (HS) vials were used. The bromination reaction of the substrate required three bromide ions to fully brominate the ketone followed by hydrolysis resulting in CHBr₃. Therefore, the bromine water was used in a slight stoichiometric excess to ensure complete bromination and to account for the disproportionation reaction of the hypobromite ion forming bromide and bromate ions. The vials were sealed using a crimp cap and a crimper, lightly shaken to ensure the mixing of the materials. The sealed vials were kept at room temperature for 24 hours.

A solution of 0.112 M sodium thiosulfate was prepared to quench any oxidized bromine species remaining in the solution. From each of the substrate reaction vials, 100 μL of the resulting solutions were drawn out with a 1 mL disposable syringe. This was injected into separate closed headspace vials containing 10 mL of the sodium thiosulfate solution. The reaction mixtures were subjected to GCMS analysis under conditions optimized for water analysis.

### Enzyme Reaction Via VHPO Mediated Pathway

The alternative substrates were tested with the addition of bromine water (i.e., HOBr) and analyzed using GCMS. Substrates that showed significant peaks for bromoform indicating a reaction with HOBr were then considered for the enzyme pathway reaction. For the positive control dimedone, 8 mL of a 0.2 M HEPES buffer (pH adjusted)/10 mM dimedone/40 mM potassium bromide (4 equivalents) solution was added to a 20 mL headspace vial. To this solution, 1 mL of VHPO solution (e.g., Curvularia sp. AU-4388 supernatant), 200 μL of 0.05 M sodium vanadate, and 70 μL of 1.16 M hydrogen peroxide were added in sequential order. Additional 0.2 M HEPES buffer (pH adjusted) was added to achieve a total volume of 10 mL.

Citric acid: A 0.2 M HEPES buffer solution was prepared, and the pH adjusted to the desired value using sodium hydroxide or hydrochloric acid. Approximately, 90 ml of the 0.2M HEPES buffer was transferred into an Erlenmeyer flask. To this, 192 mg of citric acid was measured and added to make a solution of 10 mM citric acid in 0.2M HEPES buffer, volume was adjusted later to 100 ml. From this solution approximately 8 mL of 10 mM citric acid solution was put into a 20 mL HS vial. To this, approximately 8 equivalents of a 1.16 M H₂O₂ and 0.64 mmoles of KBr solid was added and gently shaken to mix the KBr into the solution. This was followed by the addition of 1 mL of VHPO enzyme (e.g., Curvularia sp. AU-4388 supernatant) and 200 μL of 0.05 M sodium vanadate solution, additional 0.2 M HEPES buffer (pH adjusted) was added to achieve a total volume of 10 mL to make up an approximate of 8 mM citric acid solution in the HS vial. For the citric acid enzyme reaction, a small amount (less than 1 mg) of copper (II) sulphate was added in the HS vial at the end, which catalyzes the reaction (Blatchley 2003). When present in solution, Cu²¹ complexes with citrate ion as well as beta-ketoglutaric acid. Copper complexation is hypothesized to promote decarboxylation and enolization. The headspace vial was sealed using a 20 mm crimp caps and crimper. The increased concentration of KBr and H₂O₂ to 80 mM was because compared to dimedone, citric acid undergoes bromination to produce intermediates that eventually interacts with HOBr to form CHBr₃.

Methyl pyruvate: Approximately 100 mL of 0.2 M HEPES buffer (pH adjusted) was transferred into a clean Erlenmeyer flask. To prepare a 10 mM of methyl pyruvate solution in 100 mL HEPES buffer, approximately 90 μL of methyl pyruvate solution was pipetted into the Erlenmeyer flask and shaken for the purpose of mixing. From the 100 mL solution of 10 mM methyl pyruvate, 8 mL was transferred into a headspace vial. To this, approximately 8 equivalents of 1.16 M H₂O₂ and 0.64 mmoles of KBr was added and gently shaken, followed by the addition of 1 mL VHPO enzyme and 200 μL of 0.05 M sodium vanadate. 0.2 M HEPES buffer was later added to adjust the total volume of the vial to 10 mL. The vial was sealed using a 20 mm crimp caps and crimper.

The sealed vials were kept for approximately 24 hours at room temperature. After 24 hours, approximately 100 μL of each of the reaction mixtures for citric acid and methyl pyruvate was drawn out using 1 ml disposable syringe. These were then transferred into sealed HS vial containing 10 mL of 0.112M sodium thiosulfate to achieve a 100-fold dilution and subjected to a GCMS analysis along with the positive control to check for production of CHBr₃ using the appropriate method for bromoform in water analysis.

### Results

### Chemical Addition of HOBr

The GCMS was run utilizing a bromoform in water analysis method. The positive control containing dimedone generated a significant peak with both HEPES buffer and sodium citrate buffer. However, HEPES buffer was seen to be advantageous since it generated a higher GCMS peak value compared to the citrate buffer. Good conversion to bromoform was attained for dimedone, citric acid and methyl pyruvate.

### Enzyme Reaction Following VHPO Pathway

The GCMS analysis revealed a significant peak for dimedone with both buffers, indicating their reactivity with HOBr and successful production of bromoform using the fungal VHPO enzyme. However, the alternative substrates that were assesses generated negligible or no peak in the analysis when using the VHPO expressed in Curvularia sp. 4388 (NMI Accession No. V22/011149) (i.e., SEQ ID NO: 2).

### Example 8. Assessing Citric Acid as a Substrate for the VHPO Catalyzed Reaction with the Enzyme from C. pseudobrachyspora US-215 (ATCC Accession No. PTA-127509)

The enzyme reaction following the VHPO pathway described in Example 7 will be repeated with purified VHPO from C. pseudobrachyspora US-215 (ATCC Accession No. PTA-127509) (SEQ ID NO: 5). This VHPO has an active center pocket that binds citric acid with a good affinity and in a catalytically relevant pose (see Example 4). The reactions will include the substrate, citric acid, as well as dimedone, which is used as a positive control. It is anticipated that the citric acid substrate will produce a significant peak for bromoform detectable with GCMS similar to that produced by dimedone in the reaction.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

### INCORPORATION BY REFERENCE

All references, articles, publications, patents, patent publications, and patent applications cited herein within the above text and/or cited below are incorporated by reference in their entireties for all purposes. However, mention of any reference, article, publication, patent, patent publication, and patent application cited herein is not, and should not be taken as acknowledgment or any form of suggestion that they constitute valid prior art or form part of the common general knowledge in any country in the world.

### REFERENCES

Bayly, C. I., Cieplak, P., Cornell, W., and Peter A. Kollman, P. A., A well-behaved electrostatic potential based method using charge restraints for deriving atomic charges: the RESP model. The Journal of Physical Chemistry 1993 97 (40), 10269-10280.
Blatchley E R 3rd, Margetas D, Duggirala R. Copper catalysis in chloroform formation during water chlorination. Water Res. 2003 November; 37(18):4385-94.
Gérard E F, Mokkawes T, Johannissen L O, Warwicker J, Spiess R R, Blanford C F, Hay S, Heyes D J, de Visser S P. How Is Substrate Halogenation Triggered by the Vanadium Haloperoxidase from Curvularia inaequalis?ACS Catal. 2023 Jun. 6; 13(12):8247-8261.
Goel, G., Makkar, H. P. S., & Becker, K. (2009). Inhibition of methanogens by bromochloromethane: Effects on microbial communities and rumen fermentation using batch and continuous fermentations. British Journal of Nutrition, 101(10), 1484.
Hemrika, W., Renirie, R., Macedo-Ribeiro, S., Messerschmidt, A., & Wever, R. (1999). Heterologous Expression of the Vanadium-containing Chloroperoxidase from Curvularia inaequalis in Saccharomyces cerevisiae and Site-directed Mutagenesis of the Active Site Residues His496, Lys353, Arg360, and Arg490. Journal of Biological Chemistry, 274(34), 23820-23827.
Honan, M., Feng, X., Tricarico, J. M., & Kebreab, E. (2021). Feed additives as a strategic approach to reduce enteric methane production in cattle: Modes of action, effectiveness and safety. Animal Production Science.
Jumper, J., Evans, R., Pritzel, A. et al. Highly accurate protein structure prediction with AlphaFold. Nature 596, 583-589 (2021).
Kim S J, Lee J A, Kim Y H, Song B K. Optimization of the functional expression of Coprinus cinereus peroxidase in Pichia pastoris by varying the host and promoter. J Microbiol Biotechnol. 2009 September; 19(9):966-71.
Krainer F W, Gerstmann M A, Darnhofer B, Birner-Gruenberger R, Glieder A. Biotechnological advances towards an enhanced peroxidase production in Pichia pastoris. J Biotechnol. 2016 Sep. 10; 233:181-9.
Lambie, S. C., Kelly, W. J., Leahy, S. C., Li, D., Reilly, K., McAllister, T. A., Valle, E. R., Attwood, G. T., & Altermann, E. (2015). The complete genome sequence of the rumen methanogen Methanosarcina barkeri CM1. Standards in Genomic Sciences, 10(1), 57.
Macedo-Ribeiro S, Hemrika W, Renirie R, Wever R, Messerschmidt A. X-ray crystal structures of active site mutants of the vanadium-containing chloroperoxidase from the fungus Curvularia inaequalis. J Biol Inorg Chem. 1999 April; 4(2):209-19.
Machado, L., Magnusson, M., Paul, N. A., de Nys, R., & Tomkins, N. (2014). Effects of Marine and Freshwater Macroalgae on In Vitro Total Gas and Methane Production. PLoS ONE, 9(1), e85289.
Machado, L., Magnusson, M., Paul, N. A., Kinley, R., de Nys, R., & Tomkins, N. (2016). Identification of bioactives from the red seaweed Asparagopsis taxiformis that promote antimethanogenic activity in vitro. Journal of Applied Phycology, 28(5), 3117-3126.
Messerschmidt A, Wever R. X-ray structure of a vanadium-containing enzyme: chloroperoxidase from the fungus Curvularia inaequalis. Proc Natl Acad Sci USA. 1996 Jan. 9; 93(1):392-6. doi: 10.1073/pnas.93.1.392. PMID: 8552646; PMCID: PMC40244.
Messerschmidt, Albrecht, Prade, Lars and Wever, Ron. “Implications for the Catalytic Mechanism of the Vanadium-Containing Enzyme Chloroperoxidase from the Fungus Curvularia inaequalis by X-Ray Structures of the Native and Peroxide Form” Biological Chemistry, vol. 378, No. 3-4, 1997, pp. 309-316.
Morris, G. M., Goodsell, D. S., Halliday, R. S., Huey, R., Hart, W. E., Belew, R. K., Olso. A. J. J. Comput. Chem. 1998, 19, 1639-1662.
Mubarak, M. Qadri E., Gérard, Emilie F., Blanford, Christopher F., Hay, Sam, and de Visser, Sam P. How Do Vanadium Chloroperoxidases Generate Hypochlorite from Hydrogen Peroxide and Chloride?A Computational Study. ACS Catalysis 2020 10 (23), 14067-14079
Renirie, R., Hemrika, W., Piersma, S. R., & Wever, R. (2000). Cofactor and Substrate Binding to Vanadium Chloroperoxidase Determined by UV-VIS Spectroscopy and Evidence for High Affinity for Pervanadate. Biochemistry, 39(5), 1133-1141.
Roque, B. M., Brooke, C. G., Ladau, J., Polley, T., Marsh, L. J., Najafi, N., Pandey, P., Singh, L., Kinley, R., Salwen, J. K., Eloe-Fadrosh, E., Kebreab, E., & Hess, M. (2019). Effect of the macroalgae Asparagopsis taxiformis on methane production and rumen microbiome assemblage. Animal Microbiome, 1(1), 3.
Ryu K, Kang J H, Wang L, Lee E K. Expression in yeast of secreted lignin peroxidase with improved 2,4-dichlorophenol degradability by DNA shuffling. J Biotechnol. 2008 Jun. 30; 135(3):241-6.
Soedjak, H. S., Walker, J. V. & Butler, A. 1995. Inhibition and inactivation of vanadium bromoperoxidase by the substrate hydrogen peroxide and further mechanistic studies. Biochemistry, 34, 12689-12696.
Teufel F, Almagro Armenteros J J, Johansen A R, Gislason M H, Pihl S I, Tsirigos K D, Winther O, Brunak S, von Heijne G, Nielsen H. SignalP 6.0 predicts all five types of signal peptides using protein language models. Nat Biotechnol. 2022 July; 40(7):1023-1025. doi: 10.1038/s41587-021-01156-3.
Thapa, H. R., Lin, Z., Yi, D., Smith, J. E., Schmidt, E. W., & Agarwal, V. (2020). Genetic and Biochemical Reconstitution of Bromoform Biosynthesis in Asparagopsis Lends Insights into Seaweed Reactive Oxygen Species Enzymology. ACS Chemical Biology, 15(6), 1662-1670.
Wang H, Lu F, Sun Y, Du L. Heterologous expression of lignin peroxidase of Phanerochaete chrysosporium in Pichia methanolica. Biotechnol Lett. 2004 October; 26(20):1569-73.
Wen B, Baker M R, Zhao H, Cui Z, Li Q X. Expression and Characterization of Windmill Palm Tree (Trachycarpus fortunei) Peroxidase by Pichia pastoris. J Agric Food Chem. 2017 Jun. 14; 65(23):4676-4682.
Wever, R., & Hemrika, W. (2001). Vanadium haloperoxidases. Handbook of Metalloproteins. Edited by Albrecht Messerschmidt, Robert Huber, Thomas Poulos and Karl Wieghardt. John Wiley & Sons, Ltd, Chichester.
Wood, J. M., Kennedy, F. Scott., & Wolfe, R. S. (1968). Reaction of multihalogenated hydrocarbons with free and bound reduced vitamin B12. Biochemistry, 7(5), 1707-1713.

What is claimed is:

1. A method for reducing methane emissions from a ruminant comprising administering to the ruminant a composition comprising a fungal strain, biomass from the fungal strain, a culture supernatant from the fungal strain, or a combination thereof,
wherein the fungal strain comprises a vanadium-dependent haloperoxidase (VHPO) with an amino sequence having at least 80% identity with SEQ ID NO: 3, SEQ ID NO: 4, SEQ ID NO: 5, SEQ ID NO: 6, SEQ ID NO: 7, or SEQ ID NO: 8.

2. The method of claim 1, wherein the VHPO comprises SEQ ID NO: 3.

3. The method of claim 1, wherein the VHPO comprises SEQ ID NO: 4.

4. The method of claim 1, wherein the VHPO comprises SEQ ID NO: 5.

5. The method of claim 1, wherein the VHPO comprises SEQ ID NO: 6.

6. The method of claim 1, wherein the VHPO comprises SEQ ID NO: 7.

7. The method of claim 1, wherein the VHPO comprises SEQ ID NO: 8.

8. The method of any one of claims 1 to 7, wherein the VHPO demonstrates an improved property compared to a reference VHPO comprising SEQ ID NO: 1 or SEQ ID NO: 2, and
the improved property is increased substrate specificity, substrate selectivity, substrate binding, cofactor specificity, cofactor selectivity, cofactor binding, enzyme activity, enzyme kinetics, enzyme expression, enzyme stability, enzyme biocatalysis, enzyme glycosylation, enzyme methylation, enzyme acetylation, retention of the enzyme in the cell or sub-cellular component, secretion of the enzyme from the cell, and/or enzyme activity across a broader temperature range or pH range compared to the reference VHPO compared to the reference VHPO.

9. A method for reducing methane emissions from a ruminant comprising administering to the ruminant a halomethane produced with a vanadium-dependent haloperoxidase (VHPO) having an amino sequence with at least 80% identity with SEQ ID NO: 3, SEQ ID NO: 4, SEQ ID NO: 5, SEQ ID NO: 6, SEQ ID NO: 7, or SEQ ID NO: 8,
wherein the halomethane is selected from the group consisting of methyl bromide, methylene bromide, bromoform, methyl iodide, methylene iodide, iodoform, methyl chloride, methylene chloride, chloroform, and combinations thereof.

10. The method of claim 9, wherein the VHPO comprises SEQ ID NO: 3.

11. The method of claim 9, wherein the VHPO comprises SEQ ID NO: 4.

12. The method of claim 9, wherein the VHPO comprises SEQ ID NO: 5.

13. The method of claim 9, wherein the VHPO comprises SEQ ID NO: 6.

14. The method of claim 9, wherein the VHPO comprises SEQ ID NO: 7.

15. The method of claim 9, wherein the VHPO comprises SEQ ID NO: 8.

16. The method of any one of claims 9 to 15, wherein the halomethane is bromoform.

17. The method of any one of claims 9 to 16, wherein the halomethane is administered in an amount of between 1 mg/kg dry matter intake (DMI) and 1000 mg/kg DMI to the ruminant.

18. A method for reducing methane emissions from a ruminant comprising administering to the ruminant a composition comprising a fungal strain, biomass from the fungal strain, a culture supernatant from the fungal strain, or a combination thereof,
wherein the fungal strain is Curvularia lunata US-991 (ATCC Accession No. PTA-127504), Curvularia pseudobrachyspora US-215 (ATCC Accession No. PTA-127509), Curvularia spicifera US-188 (ATCC Accession No. PTA-127510), Curvularia inaequalis US-998 (ATCC Accession No. PTA-127511), Curvularia intermedia US-429 (ATCC Accession No. PTA-127512), Curvularia hawaiiensis US-446 (ATCC Accession No. PTA-127601), Curvularia protuberata US-1111 (ATCC Accession No. PTA-127607), or a mutant thereof having all identifying characteristics of the strain.

19. The method of claim 18, wherein the fungal strain is Curvularia lunata US-991 (ATCC Accession No. PTA-127504) or a mutant thereof having all identifying characteristics of the strain.

20. The method of claim 18, wherein the fungal strain is Curvularia pseudobrachyspora US-215 (ATCC Accession No. PTA-127509) or a mutant thereof having all identifying characteristics of the strain.

21. The method of claim 18, wherein the fungal strain is Curvularia spicifera US-188 (ATCC Accession No. PTA-127510) or a mutant thereof having all identifying characteristics of the strain.

22. The method of claim 18, wherein the fungal strain is Curvularia inaequalis US-998 (ATCC Accession No. PTA-127511) or a mutant thereof having all identifying characteristics of the strain.

23. The method of claim 18, wherein the fungal strain is Curvularia intermedia US-429 (ATCC Accession No. PTA-127512) or a mutant thereof having all identifying characteristics of the strain.

24. The method of claim 18, wherein the fungal strain is Curvularia hawaiiensis US-446 (ATCC Accession No. PTA-127601) or a mutant thereof having all identifying characteristics of the strain.

25. The method of claim 18, wherein the fungal strain is Curvularia protuberata US-1111 (ATCC Accession No. PTA-127607) or a mutant thereof having all identifying characteristics of the strain.

26. A method for reducing methane emissions from a ruminant comprising administering to the ruminant a halomethane produced by a fungal strain selected from the group consisting of Curvularia lunata US-991 (ATCC Accession No. PTA-127504), Curvularia pseudobrachyspora US-215 (ATCC Accession No. PTA-127509), Curvularia spicifera US-188 (ATCC Accession No. PTA-127510), Curvularia inaequalis US-998 (ATCC Accession No. PTA-127511), Curvularia intermedia US-429 (ATCC Accession No. PTA-127512), Curvularia hawaiiensis US-446 (ATCC Accession No. PTA-127601), Curvularia protuberata US-1111 (ATCC Accession No. PTA-127607), and a mutant thereof having all identifying characteristics of the strain,
wherein the halomethane is selected from the group consisting of methyl bromide, methylene bromide, bromoform, methyl iodide, methylene iodide, iodoform, methyl chloride, methylene chloride, chloroform, and combinations thereof.

27. The method of claim 26, wherein the fungal strain is Curvularia lunata US-991 (ATCC Accession No. PTA-127504) or a mutant thereof having all identifying characteristics of the strain.

28. The method of claim 26, wherein the fungal strain is Curvularia pseudobrachyspora US-215 (ATCC Accession No. PTA-127509) or a mutant thereof having all identifying characteristics of the strain.

29. The method of claim 26, wherein the fungal strain is Curvularia spicifera US-188 (ATCC Accession No. PTA-127510) or a mutant thereof having all identifying characteristics of the strain.

30. The method of claim 26, wherein the fungal strain is Curvularia inaequalis US-998 (ATCC Accession No. PTA-127511) or a mutant thereof having all identifying characteristics of the strain.

31. The method of claim 26, wherein the fungal strain is Curvularia intermedia US-429 (ATCC Accession No. PTA-127512) or a mutant thereof having all identifying characteristics of the strain.

32. The method of claim 26, wherein the fungal strain is Curvularia hawaiiensis US-446 (ATCC Accession No. PTA-127601) or a mutant thereof having all identifying characteristics of the strain.

33. The method of claim 26, wherein the fungal strain is Curvularia protuberata US-1111 (ATCC Accession No. PTA-127607) or a mutant thereof having all identifying characteristics of the strain.

34. The method of any one of claims 26 to 33, wherein the halomethane is bromoform.

35. The method of any one of claims 26 to 34, wherein the halomethane is administered in an amount of between 1 mg/kg dry matter intake (DMI) and 1000 mg/kg DMI to the ruminant.

36. The method of any one of claims 1 to 35, wherein the ruminant is a member of the family Bovidae.

37. The method of claim 36, wherein the ruminant is Bos taurus or Bos indicus.

38. The method of claim 36, wherein the ruminant is Ovis aries or Capra hircus.

39. A cell or a biologically pure culture of a fungal strain comprising a VHPO with an amino sequence having at least 80% identity with SEQ ID NO: 3, SEQ ID NO: 4, SEQ ID NO: 5, SEQ ID NO: 6, SEQ ID NO: 7, or SEQ ID NO: 8.

40. The cell or a biologically pure culture of claim 39, wherein the fungal strain is a yeast belonging to the genus of Arxula, Candida, Ogataea, Kluyveromyces, Pichia, Saccharomyces, or Yarrowia.

41. The cell or a biologically pure culture of claim 40, wherein the yeast is Saccharomyces cerevisiae, Yarrowia lipolytica, or Pichia pastoris.

42. A cell or a biologically pure culture of Curvularia lunata US-991 (ATCC Accession No. PTA-127504), Curvularia pseudobrachyspora US-215 (ATCC Accession No. PTA-127509), Curvularia spicifera US-188 (ATCC Accession No. PTA-127510), Curvularia inaequalis US-998 (ATCC Accession No. PTA-127511), Curvularia intermedia US-429 (ATCC Accession No. PTA-127512), Curvularia hawaiiensis US-446 (ATCC Accession No. PTA-127601), Curvularia protuberata US-1111 (ATCC Accession No. PTA-127607), or a mutant thereof having all identifying characteristics of the strain.

43. An agricultural composition comprising the cell or a biologically pure culture of any one of claims 39 to 42 or biomass from the cell or biologically pure culture, a culture supernatant from the cell or biologically pure culture, or a combination thereof, and,
optionally, an agriculturally acceptable carrier.

44. The agricultural composition of claim 43, further comprising a cereal, starch, vegetable waste, vitamin, mineral, trace element, emulsifier, aromatizing product, binder, colorant, odorant, thickening agent, or a combination thereof.

45. Animal feed comprising the biologically pure culture of any one of claims 39 to 42 or the agricultural composition of claim 43 or 44.

46. The animal feed of claim 45, wherein the animal feed is in the form of a powder, granule, pellet, feed block, or lick block.

47. Use of the cell or the biologically pure culture of any one of claims 39 to 42, the agricultural composition of claim 43 or 44, or the animal feed of claim 45 or 46 for reducing methane emissions from a ruminant.

48. The use of claim 47, wherein the ruminant is a member of the family Bovidae.

49. The use of claim 48, wherein the ruminant is Bos taurus or Bos indicus.

50. The use of claim 48, wherein the ruminant is Ovis aries or Capra hircus.
